(19) United States
(12) Patent Application Publication
(10) Pub. No.: US 2016/0158333 A1
(43) Pub. Date: Jun. 9, 2016

(54) METHODS FOR PRODUCING SALMONELLA O-ANTIGEN CAPSULES, COMPOSITIONS AND USES THEREOF

(71) Applicants: Aaron P. WHITE, Saskatoon, Saskatchewan (CA); Sumundu R. PERERA, Saskatoon, Saskatchewan (CA); Shirley LAM, Saskatoon (CA); Deanna L. GIBSON, Saskatoon, Saskatchewan (CA); UNIVERSITY OF SASKATCHEWAN, Saskatchewan (CA)

(72) Inventors: Aaron P. White, Saskatoon, Saskatchewan (CA); Sumundu R. Perera, Saskatoon, Saskatchewan (CA); Shirley Lam, Saskatoon, Saskatchewan (CA); Deanna L. Gibson, Saskatoon, Saskatchewan (CA)

(73) Assignees: University of Saskatchewan, Saskatoon, SK (CA); Universiyt of British Columbia, Kelowna, BC (CA)

(21) Appl. No.: 14/907,681

(22) PCT Filed: Jul. 25, 2014
(86) PCT No.: PCT/CA2014/000582

§ 371 Date: Jan. 26, 2016

Related U.S. Application Data
(60) Provisional application No. 61/858,969, filed on Jul. 26, 2013.
(60) Provisional application No. 61/887,186, filed on Oct. 4, 2013.

(51) Int. Cl. A61K 39/112; C12P 19/04; G01N 33/68; A61K 39/40

(52) U.S. Cl. CPC A61K 39/0275; A61K 39/40; C12P 19/04; G01N 33/6854; G01N 2333/255; G01N 2469/20

(57) ABSTRACT

Methods of purifying O-Ag capsules from an S. enterica NTS serovar, wherein the O-Ag capsule is substantially free of co-expressed cellulose and LPS, are described, as are immunogenic compositions comprising the O-Ag capsules and methods for treating, preventing and diagnosing Salmonella infections. Also described are constructs and methods for producing S. enterica NTS serovar mutants which over-express the O-Ag capsule.

### TECHNICAL FIELD

The present invention pertains generally to immunogenic compositions and methods for treating and/or preventing Salmonella infection. In particular, the invention relates to the production, purification and use of an O-antigen capsular polysaccharide (“O-Ag”) in compositions for the treatment and/or prevention of non-typhoidal Salmonella infections.

### BACKGROUND

Salmonella enterica are Gram negative enteropathogenic bacteria. Within the S. enterica species, more than 2300 serovars have been identified, many of which cause gastroenteritis and are collectively referred to as non-typhoidal Salmonella (NTS). Serovars Enteritidis (S. Enteritidis), Typhimurium (S. Typhimurium) and Heidelberg (S. Heidelberg) have been the most frequently associated with human infections such as gastroenteritis. S. Enteritidis is a well known zoonotic pathogen (Imke Hansen-Wester, M. H., Microbes and Infection (2001) 3:549-559) and poultry infected with this pathogen are among the most common reservoir of salmonellae that can be transmitted through the food chain to humans (Gask, R. K., 2003. Salmonella infections, p. 567. In Y. M. Saif, H. J. Barnes, J. R. Glisson, A. M. Fadly, and L. R. McDougald (ed.), Diseases of Poultry, 11th ed., Iowa State Press).

One of the main sources of NTS contraction is international travel, especially to endemic areas in the developing world. Thus, the development of a vaccine for NTS-caused gastroenteritis would be highly desirable. A vaccine against NTS-induced gastroenteritis would reduce the prevalence of Salmonella infections which can lead to life threatening infections in immunocompromised individuals (e.g., young, elderly, cancer and HIV-positive individuals). Such a vaccine would also find use for individuals visiting or residing in endemic areas of the world.

Many pathogenic bacteria, including Salmonella, produce capsular polysaccharides (CPS). CPS can mediate diverse functions, such as resistance to antimicrobials and defense against the host immune system through prevention of phagocytosis. In addition, CPS play a role in the formation of biofilms where they can provide nutrient sequestration and desiccation resistance. CPS are typically bound to the bacterial surface due to the presence of fatty acids that anchor the capsule to the cell. Lipopolysaccharide (LPS) represents the most dominant type of polysaccharide in Gram-negative bacteria, where the oligosaccharide chain, termed the O antigen, is linked to a defined lipid A core within the outer membrane.

CPS have been divided into four groups based on size, charge density, assembly mechanism and means of attachment to the cell surface. Group 4 capsules are comprised of repeat units that are structurally identical to the oligosaccharide units in LPS, and hence are referred to as O-antigen (O-Ag) capsules. Despite having structurally similar repeat units as LPS, O-Ag capsules use a distinct translocation system and typically are long chains comprised of hundreds of repeat units.

In Salmonella, only a few polysaccharides have been identified. Colanic acid is an anionic polymer produced exclusively at low temperatures (i.e., 24° C.) and has no known role in virulence. Cellulose was more recently discovered, is ubiquitous throughout Salmonella, and plays a primary role in biofilm formation and resistance to disinfection; it also has no known role in virulence. The most well characterized Salmonella extracellular polysaccharide is the Vi antigen, which is found almost exclusively in S. enterica serovar Typhi. Purified Vi forms the basis of the injectable vaccine for typhoid fever (Crump et al. (2010) Clin. Infect. Dis. 50:241-246). Two additional CPS have been identified in Salmonella, the O-Ag capsule (Gibson et al., (2006) J Bacteriol. 188:7722-7730) and an anionic polymer with a unique chemical composition that has not been well characterized (de Rezende et al. (2005) Appl. Environ. Microbiol. 71:7345-7351).

The O-Ag capsule was first purified from Salmonella ser. Enteritidis 27655-3b (hereafter referred to as S. Enteritidis 3b) (Gibson et al., (2006) J. Bacteriol. 188:7722-7730). Structural determination showed it had a repeat unit nearly identical to the LPS O-antigen (FIGS. 1 A-C). Despite the similar repeat units, the O-Ag capsule differs from LPS in several ways. In particular, the O-Ag capsule (1) is not associated with the LPS core region, (2) has a lower net charge, therefore can be separated by anion exchange chromatography, (3) is partially substituted with glucose on the tyvelose side chain (FIG. 1B, Tyv), whereas LPS only has this modification on galactose (FIG. 1C, Gal), and (4) is comprised of >3000 repeat units as compared to ˜20-30 repeats in LPS. The yih operons (FIG. 1D), responsible for O-Ag capsule biosynthesis, were identified by screening a S. Enteritidis 3b transposon library with capsule-specific immune serum. Mutation of yihO or yihQ yielded capsule-negative strains. Immune serum raised to purified O-Ag capsule has only minimal cross-reactivity to intact LPS.

PCR screening and analysis of sequenced Salmonella genomes indicates that the yihUTSRQPO and yihVW operons are present in isolates from all seven S. enterica subspecies. Thus, it appears the O-Ag capsule assembly and translocation machinery is conserved in all subgroups (Gibson et al., (2006) J. Bacteriol. 188:7722-7730). Cross-reactive capsular material has also been detected in these isolates, as tested by ELISA using O-Ag capsule-specific serum which indicates that O-Ag capsule structures from diverse S. enterica isolates are immunologically cross-reactive.

It has been demonstrated that the O-Ag capsule is critical for the desiccation resistance of S. Enteritidis 3b (Gibson et al., (2006) J. Bacteriol. 188:7722-7730). The capsule is produced as part of the extracellular matrix of the rdar morphotype, a colony morphology (red, dry, and rough) that most S. enterica isolates are able to form. Many studies have shown that the rdar morphotype is a conserved survival mechanism, mediating persistence of Salmonella in the face of disinfectants and harsh environmental conditions and O-Ag has been postulated to play a role in Salmonella virulence and/or interactions with the host immune system (White et al. (2008) Infect. Immun. 76:1048-1058).

The O-Ag capsule has also been shown to aid in the ability of a S. enterica isolate to stick to plant surfaces (Barak et al. (2007) Mol. Plant Microbe Interact. 20:1083-1091), as well as being involved in the colonization of gallstones by Salmonella ser. Typhi (Crawford et al. (2008) Infect. Immun. 76:5341-5349; Crawford et al. (2010) Proc. Natl. Acad. Sci. USA 107:4353-4358).

However, the use of the O-Ag capsule as a vaccine antigen has not heretofore been suggested. There remains a need for the development of effective strategies for the treatment, prevention and diagnosis of NTS infection.

### SUMMARY OF THE INVENTION

The present invention is based on the discovery of effective production and purification methods of the O-Ag capsule, as well as methods to isolate the O-Ag capsule away from co-expressed cellulose and lipopolysaccharide (LPS). Isolation from the LPS is particularly desirable as injection of even a small amount of LPS has been shown to be pyrogenic, cause a decrease in blood pressure, and activate inflammation and coagulation. LPS endotoxins are in large part responsible for the dramatic clinical manifestations of infections with pathogenic Gram-negative bacteria.

Moreover, production of the O-Ag capsule is increased by genetically knocking out transcriptional modifiers as described further herein.

Thus, the O-Ag capsule produced and purified as described herein is useful in vaccine compositions for the treatment and/or prevention of NTS infection. Such a vaccine can reduce the prevalence of Salmonella infections which can lead to life threatening infections in immunocompromised individuals (e.g., young, elderly, cancer and HIV-positive individuals) and also finds use in individuals visiting or residing in endemic areas of the world.

Accordingly, in one embodiment, the invention is directed to a method of preparing an NTS O-Ag capsule preparation comprising purifying the O-Ag capsule from an S. enterica NTS serovar, wherein the O-Ag capsule is substantially free of co-expressed cellulose and LPS.

In additional embodiments, the O-Ag capsule is prepared by a method that comprises:
(a) providing a cellulose-deficient S. enterica NTS serovar mutant;
(b) isolating cell surface components from the S. enterica NTS serovar, wherein the cell surface components comprise the O-Ag capsule;
(c) applying the cell surface components to an anion exchange chromatography column under conditions whereby fractions comprising the O-Ag capsule are eluted;
(d) applying O-Ag capsule-containing fractions to a size-exclusion chromatography column under conditions whereby fractions comprising the O-Ag capsule are eluted;
(e) collecting fractions that include the O-Ag capsule;
(f) performing phase separation on the O-Ag capsule-containing fractions under conditions that separate LPS from the O-Ag capsule;
to provide O-Ag capsule substantially free of co-expressed cellulose and LPS.

In certain embodiments, step (f) is performed using a polyethylene glycol detergent.

In additional embodiments, the amount of LPS remaining in the final product is under 2×10⁵ EU/mg.

In additional embodiments, the invention is directed to a composition comprising an immunogenic S. enterica NTS O-Ag capsule, wherein the S. enterica NTS O-Ag capsule is prepared by any of the methods above.

In yet further embodiments, the invention is directed to a composition comprising a pharmaceutically acceptable vehicle and (a) an immunogenic S. enterica NTS O-Ag capsule, wherein the O-Ag capsule is substantially free of co-expressed cellulose and LPS; (b) an immunogenic fragment of (a), or (c) antibodies reactive with the O-Ag capsule.

In additional embodiments, the invention is directed to a method of producing an immunogenic composition comprising (a) providing a purified, immunogenic S. enterica NTS O-Ag capsule; and (b) combining said purified O-Ag capsule with a pharmaceutically acceptable vehicle.

In certain embodiments, the S. enterica NTS O-Ag capsule is prepared by any one of the methods above.

In further embodiments, the invention is directed to a method of treating or preventing an S. enterica NTS infection in a vertebrate subject comprising administering to the subject a therapeutically effective amount of a composition as described above.

In additional embodiments, the invention is directed to a method of reducing the amount of S. enterica NTS in the intestinal tract of a vertebrate subject comprising administering to the subject a therapeutically effective amount of a composition as described above.

In certain embodiments of the above methods, the vertebrate subject is an avian or a mammalian subject, such as a human.

In yet further embodiments the invention is directed to a method of detecting S. enterica NTS antibodies in a biological sample, comprising:
(a) reacting the biological sample with an immunogenic S. enterica NTS O-Ag capsule, under conditions which allow NTS antibodies, when present in the biological sample, to bind to the capsule to form an antibody/antigen complex; and
(b) detecting the presence or absence of the complex, and thereby detecting the presence or absence of S. enterica NTS antibodies in the sample.

In yet additional embodiments, the invention is directed to an immunodiagnostic test kit for detecting S. enterica NTS infection, the test kit comprising an immunogenic S. enterica NTS O-Ag capsule and instructions for conducting the immunodiagnostic test.

In additional embodiments, the invention is directed to a polynucleotide encoding an S. enterica NTS serovar mutant, wherein the mutant comprises a deletion of all or a portion of the yihV and/or yihW genes of the O-Ag capsule operon, such that when the polynucleotide is expressed, O-Ag capsule production is enhanced as compared to O-Ag capsule production when yihVW remains intact. In certain embodiments, the mutant comprises a deletion of the nucleotide sequence encoding the DNA-binding region of YihW.

In further embodiments, the polynucleotide further comprises a deletion of the gene coding for cellulose synthase.

In additional embodiments, the invention is directed to a recombinant construct comprising a polynucleotide as described above, and control elements that are operably linked to the polynucleotide whereby coding sequences in the polynucleotide can be transcribed and translated in a host cell.

In yet further embodiments, the invention is directed to a host cell transformed with the recombinant construct, as well as methods of producing an O-Ag capsule comprising providing a population of such host cells and culturing the population of cells under conditions whereby the O-Ag capsule is produced. In certain embodiments, the invention is directed to further purifying the produced O-Ag capsule to provide an O-Ag capsule preparation, wherein O-Ag capsule is purified under conditions wherein the O-Ag capsule preparation is substantially free of co-expressed cellulose and LPS.

In any of the above embodiments, the S. enterica NTS serovar is selected from serovar Enteritidis (S. Enteritidis), serovar Typhimurium (S. Typhimurium) or serovar Heidelberg (S. Heidelberg).

These and other embodiments of the subject invention will readily occur to those of skill in the art in view of the disclosure herein.

### BRIEF DESCRIPTION OF THE FIGURES

FIGS. 1A-1D show the structure of the O-Ag capsule and operons required for assembly and translocation. FIG. 1A shows the configuration of the S. Enteritidis O-Ag capsule; FIG. 1B shows the linear unit from the S. Enteritidis O-Ag capsule with R representing a non-stoichiometric glucose substitution; FIG. 1C is a linear drawing of the O-antigen repeat unit from S. Enteritidis LPS; FIG. 1D depicts the yihU-O yshA and yihVW gene clusters, shown to scale. Putative protein functions are listed below each gene. The asterisk denotes the transposon insertion site used for identification of the operon. Figure adapted from Gibson et al., (2006) J. Bacteriol. 188:7722-7730.

FIG. 2 is a diagram of the reporter plasmid pCS26-Pac.

FIGS. 3A-3B show promoter activity of yihU in S. Typhimurium ΔbcsA, S. Typhimurium ΔbcsA pBR322-yihVW, S. Typhimurium ΔbcsA ΔyihVW, and S. Typhimurium ΔbcsA ΔyihVW pBR322-yihVW strains grown on 1% Tryptone.

FIGS. 4A-4B (SEQ ID NO:5) show the nucleotide sequence of a representative S. enterica NTS serovar (S. Typhimurium str. LT2) yihVW region. The coding sequence for yihV begins at nucleotide position 92 and ends at nucleotide position 988. The coding sequence for yihW begins at nucleotide position 1022 and ends at position 1825. The bolded region represents the deletion present in a representative yihVW mutant (corresponding to positions 96-1822).

FIG. 5 (SEQ ID NO:6) shows the amino acid sequence of YihV encoded by the nucleotide sequence shown in FIG. 4.

FIG. 6 (SEQ ID NO:7) shows the amino acid sequence of YihW encoded by the nucleotide sequence shown in FIG. 4.

### DETAILED DESCRIPTION OF THE INVENTION

The practice of the present invention will employ, unless otherwise indicated, conventional methods of microbiology, chemistry, polysaccharide chemistry, biochemistry, immunology, molecular biology and recombinant DNA technology within the skill of the art. Such techniques are explained fully in the literature. See, e.g., Varki A, Cummings R, Esko J, Freeze H, Stanley P, Bertozzi C, Hart G, Etzler M (2008). Essentials of Glycobiology (Cold Spring Harbor Laboratory Press; Medical Microbiology (Baron S. ed Galveston, Tex.) Handbook of Experimental Immunology, Vols. I-IV (D. M. Weir and C. C. Blackwell eds., Blackwell Scientific Publications); A.L. Lehninger, Biochemistry (Worth Publishers, Inc.); Remington's Pharmaceutical Sciences, (Easton, Pa.: Mack Publishing Company); Sambrook, Fritsch & Maniatis, Molecular Cloning: A Laboratory Manual; Perbal, B., A Practical Guide to Molecular Cloning.

All publications, patents and patent applications cited herein, whether supra or infra, are hereby incorporated by reference in their entireties.

### 1. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antigen” includes a mixture of two or more such antigens, and the like.

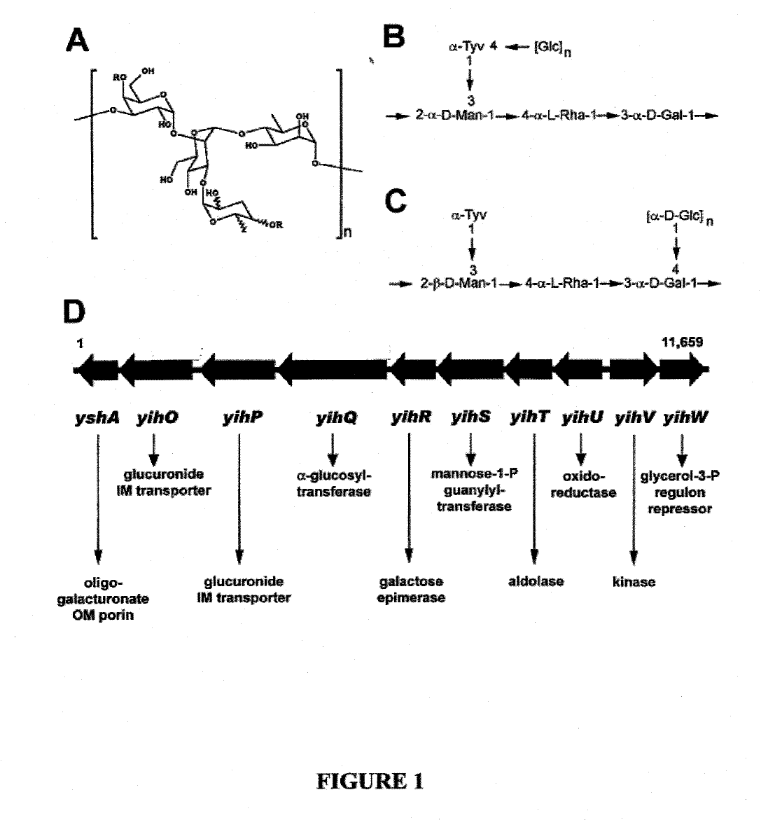

As used herein, the term NTS O-Ag capsule intends an O-Ag capsule from any of the several S. enterica NTS serovars including without limitation, S. Enteritidis, S. Typhimurium, S. Heidelberg, S. Salamae, S. Arizonae, S. Diarizonae, S. Houtenae, S. Indica, S. Dublin, etc. See, Gibson et al., (2006) J Bacteria 188:7722-7730 and FIG. 1 herein, depicting the structure of a representative S. Enteritidis O-Ag capsule. However, an O-Ag capsule, as defined herein is not limited to that shown and described as various isolates of the NTS serovars are known and variations in structure of the O-Ag capsule may occur between them. Moreover, the derived O-Ag capsule need not be physically derived from the particular NTS serovar or isolate, but may be generated in any manner, including for example, chemical synthesis or isolation, based on the information provided herein and known in the art.

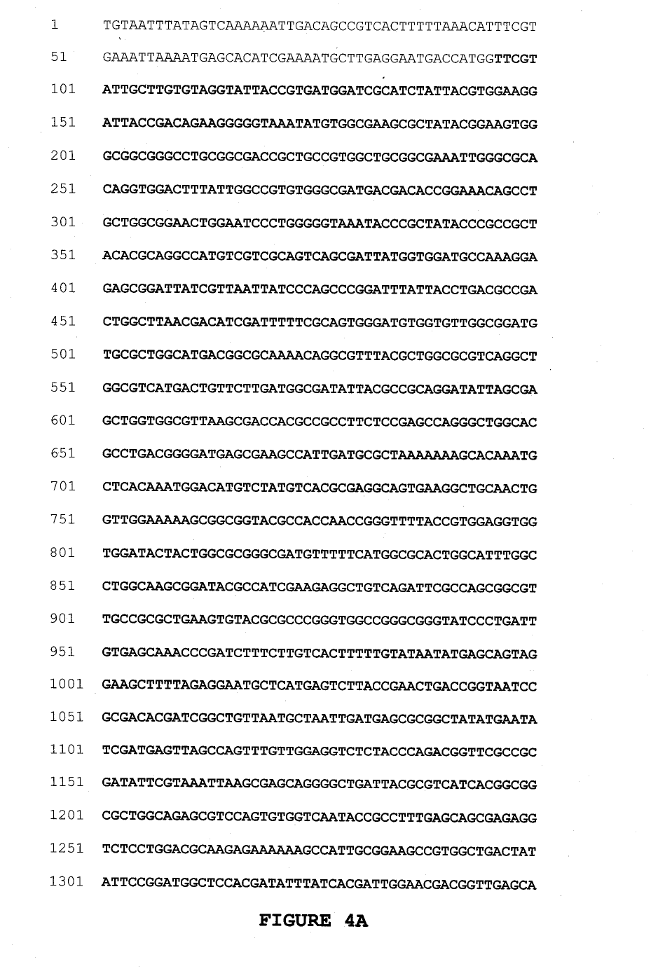

As used herein, the term “vihVW region” in reference to the region present in an S. enterica serovar refers to a vihVW region shown in the reference sequence of FIGS. 4A-4B (SEQ ID N0:5) or the corresponding positions in vihVW regions derived from any serovar, type or strain of an S. enterica NTS serovar.

As used herein, the term “vihV” in reference to the region present in an S. enterica serovar refers to a vihV region shown in the reference sequence of FIGS. 4A-4B (SEQ ID NO:5) or the corresponding positions in vihV regions derived from any serovar, type or strain of an S. enterica NTS serovar.

As used herein, the term “vihW” in reference to the region present in an S. enterica serovar refers to a vihW region shown in the reference sequence of FIGS. 4A-4B (SEQ ID N0:5) or the corresponding positions in vihV regions derived from any serovar, type or strain of an S. enterica NTS serovar. The terms “variant” and “mutant” of an O-Ag capsule refer to biologically active derivatives of the O-Ag capsule, or fragments of such derivatives, that retain immunological activity as defined herein. Preferably, the variant or mutant has at least the same activity as the native molecule. For example, the number of repeats of the monomeric units present in the molecule may vary, so long as the O-Ag retains immunogenicity. Methods for making variants and mutants are known in the art. The term “mutant” when referring to an S. enterica NTS serovar denotes a bacterium with deletions, insertions or substitutions in the genome. As described further below, typically such mutants will include deletions of all or a portion of the vihV and/or vihW regions to boost production of the O-Ag capsule, and/or deletions of the gene encoding for cellulose synthase to produce cellulose-deficient mutants.

By “immunogenic” molecule is meant a molecule which includes one or more epitopes and thus can modulate an immune response. Such molecules can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, polysaccharide epitope mapping techniques are known in the art and include those described in, e.g., Kooistra et al. (2002) European J. Biochem. 269: 573-582; Johnson et al. (2004) Biorganic Med. Chem. 12:295-300. As used herein, the term “epitope” generally refers to the site on an antigen which is recognized by a T-cell receptor and/or an antibody. Several different epitopes may be carried by a single antigenic molecule.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

Thus, an immunological response as used herein may be one that stimulates the production of antibodies. The antigen of interest may also elicit production of CTLs. Hence, an immunological response may include one or more of the following effects: the production of antibodies by B-cells; and/or the activation of suppressor T-cells and/or memory/effector T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. (See, e.g., Montefiori et al. (1988) J. Clin Microbiol. 26:231-235; Dreyer et al. (1999) AIDS Res Hum Retroviruses (1999) 15(17):1563-1571). The innate immune system of mammals also recognizes and responds to molecular features of pathogenic organisms via activation of Toll-like receptors and similar receptor molecules on immune cells. Upon activation of the innate immune system, various non-adaptive immune response cells. are activated to, e.g., produce various cytokines, lymphokines and chemokines. Cells activated by an innate immune response include immature and mature Dendritic cells of the monocyte and plasmacytoid lineage (MDC, PDC), as well as gamma, delta, alpha and beta T cells and B cells and the like. Thus, the present invention also contemplates an immune response wherein the immune response involves both an innate and adaptive response.

An “immunogenic composition” is a composition that comprises an immunogenic molecule where administration of the composition to a subject results in the development in the subject of a humoral and/or a cellular immune response to the molecule of interest.

By “subunit vaccine” is meant a vaccine composition that includes one or more selected antigens but not all antigens, derived from or homologous to, an antigen from a pathogen of interest. Such a composition is substantially free of intact pathogen cells or pathogenic particles, or the lysate of such cells or particles. Thus, a “subunit vaccine” can be prepared from at least partially purified (preferably substantially purified) immunogenic molecules from the pathogen, or analogs thereof. The method of obtaining an antigen included in the subunit vaccine can thus include standard purification techniques, recombinant production, or synthetic production.

An “antigen” refers to a molecule containing one or more epitopes (either linear, conformational or both) that will stimulate a host's immune-system to make a humoral and/or cellular antigen-specific response. The term is used interchangeably with the term “immunogen.” Antibodies such as anti-idiotype antibodies, or fragments thereof, and synthetic peptide mimotopes, which can mimic an antigen or antigenic determinant, are also captured under the definition of antigen as used herein.

“Substantially purified” generally refers to isolation of a substance such that the substance comprises the majority percent of the sample in which it resides. Typically in a sample, a substantially purified component comprises 50%, preferably 80%-85%, more preferably 90-95% of the sample. Techniques for purifying molecules of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. The term “antibody” as used herein includes antibodies obtained from both polyclonal and monoclonal preparations, as well as, the following: hybrid (chimeric) antibody molecules (see, for example, Winter et al., Nature (1991) 349:293-299; and U.S. Patent No. 4,816,567); F(ab′)2 and F(ab) fragments; Fv molecules (non-covalent heterodimers, see, for example, Inbar et al., Proc Nati Acad Sci USA (1972) 69:2659-2662; and Ehrlich et al., Biochem (1980) 19:4091-4096); single-chain Fv molecules (sFv) (see, for example, Huston et al., Proc Nad Acad Sci USA (1988) 85:5879-5883); dimeric and trimeric antibody fragment constructs; minibodies (see, e.g., Pack et al., Biochem (1992) 31:1579-1584; Cumber et al., J Immunology (1992) 149B:120-126); humanized antibody molecules (see, for example, Riechmann et al., Nature (1988) 332:323-327; Verhoeyan et al., Science (1988) 239:1534-1536; and U.K. Patent Publication No. GB 2,276,169, published 21 September 1994); and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

The term “derived from” is used herein to identify the original source of a molecule but is not meant to limit the method by which the molecule is made which can be, for example, by chemical synthesis or recombinant means.

“Native” proteins or polypeptides refer to proteins or polypeptides isolated from the source in which the proteins naturally occur. “Recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

“Homology” refers to the percent identity between two polynucleotide or two polypeptide moieties. Two nucleic acid, or two polypeptide sequences are “substantially homologous” to each other when the sequences exhibit at least about 50% , preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions =50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, bacterial, semisynthetic, or synthetic origin which, by virtue of its origin or manipulation is not associated with all or a portion of the polynucleotide with which it is associated in nature.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

A “nucleic acid” molecule or “polynucleotide” can include, but is not limited to, prokaryotic sequences, eucaryotic mRNA, cDNA from eucaryotic mRNA, genomic DNA sequences from eucaryotic (e.g., mammalian) DNA, and even synthetic DNA sequences. The term also captures sequences that include any of the known base analogs of DNA and RNA.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

A “vector” is capable of transferring gene sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; non-domestic animals such as elk, deer, mink and feral cats; laboratory animals including rodents such as mice, rats and guinea pigs; birds, including domestic, wild and game birds such as chickens, turkeys and other gallinaceous birds, ducks, geese, pheasant, emu, ostrich and the like. The term does not denote a particular age. Thus, both adult and newborn individuals are intended to be covered.

By “therapeutically effective amount” in the context of the immunogenic compositions is meant an amount of an immunogen which will induce an immunological response, either for antibody production or for treatment or prevention of infection.

As used herein, “treatment” refers to any of (i) the prevention of infection or reinfection, as in a traditional vaccine, or (ii) the reduction or elimination of symptoms from an infected individual. Treatment may be effected prophylactically (prior to infection) or therapeutically (following infection). Additionally, prevention or treatment in the context of the present invention can be a reduction of the amount of NTS S. Enterica in the intestinal tract, thus reducing transmission of disease by reducing the amount of fecal shedding of bacteria. Thus, an asymptomatic subject is still considered to have been “treated” if the amount of S. Enterica in the intestinal tract is reduced.

By “NTS disease” or “NTS infection” is meant, without limitation, salmonellosis in any of its several forms, varying from a self-limiting gastroenteritis to septicemia, bacteremia, meningitis, and the like. Whether the organism remains in the intestine or disseminates depends on host factors as well as the virulence of the strain. Asymptomatic infections are also included.

### 2. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based in part on the discovery of a purification procedure that produces the O-Ag capsule in an isolated form to be incorporated into a pharmaceutical composition, such as a vaccine composition. In particular, the inventors herein have discovered a method for purifying the O-Ag capsule away from the LPS component. Such a preparation is highly desirable as injection of a small amount of LPS endotoxin has been shown to produce fever, a decrease in blood pressure, and activation of inflammation and coagulation. Endotoxins are in large part responsible for the dramatic clinical manifestations of infections with pathogenic Gram-negative bacteria. Therefore, in pharmaceutical production, it is necessary to remove LPS endotoxins to prevent illness in humans. Moreover, drug regulatory agencies, such as the United States Food and Drug Administration (FDA), have specified limits on the amounts of LPS endotoxins that can be present in licensed products.

Additionally, the inventors herein have identified transcriptional modifiers of the O-Ag capsule production pathway. In particular, deletion of the yihVW gene increased production of the O-Ag capsule. Accordingly, enhanced amounts, as much as 1000 times or more over amounts produced when the yihVW genes remain intact, can be produced using the methods described herein.

The present invention thus provides constructs and efficient modes for producing immunological compositions and methods for treating and/or preventing NTS S. enterica disease. Immunization can be achieved by any of the methods known in the art including, but not limited to, use of vaccines containing the O-Ag capsule, or by passive immunization using antibodies directed against the O-Ag capsule. Such methods are described in detail below. Moreover, the O-Ag capsule and antibodies described herein can be used for detecting the presence of NTS S. Enterica serovars, for example in a biological sample.

The vaccines are useful in vertebrate subjects that are susceptible to NTS S. Enterica infection, including without limitation, humans, avian species, and those species that are raised for meat or egg production such as, but not limited to, chickens turkeys, geese, ducks, pheasant, emu and ostrich. Thus, the vaccines can prevent S. Enterica infection in humans caused by coming in contact with infected subjects.

In order to further an understanding of the invention, a more detailed discussion is provided below regarding the S. Enterica O-Ag capsule, production thereof, compositions comprising the same, and methods of using such compositions in the treatment or prevention of infection, as well as in the diagnosis of infection.

### A. S. Enterica O-Ag Capsule

The O-Ag capsule is an oligosaccharide chain, linked to a defined lipid A core within the outer membrane of S. enterica. The O-Ag capsule was first detected (but not identified as such) in S. Enteritidis by immunizing rabbits with a whole-cell fimbrial preparation and using the resulting immune serum on Western blots to screen whole cell lysates of S. Enteritidis. In addition to a major fimbrial subunit that migrated at 17,000 Daltons, a high molecular weight substance (>100 kDa) was detected. This high molecular weight band was assumed to represent fimbriae that were not fully depolymerized but subsequent work showed that the high molecular weight material was not fimbriae but rather a polysaccharide (White et al. (2003) J. Bacteriol 185:5398-5407). The polysaccharide component of the purported fimbrial preparation was later discovered to include two distinct polysaccharides, one that was termed the O-Ag capsule and the other which remains to be fully characterized.

The O-Ag capsule was later purified from S. Enteritidis 27655-3b (hereafter referred to as S. Enteritidis 3b) as described in Gibson et al., (2006) J. Bacteriol. 188:7722-7730 and structural determination showed it has a repeat unit nearly identical to the LPS 0-antigen (FIGS. 1A-C). However, despite the similar repeat units, the O-Ag capsule differs from LPS in several ways as it is not associated with the LPS core region, has a lower net charge and can therefore be separated by anion exchange chromatography, and is partially substituted with glucose on the tyvelose side chain (FIG. 1B, Tyv), whereas LPS only has this modification on galactose (FIG. 1C, Gal). The O-Ag capsule as isolated is comprised of >3000 repeat units as compared to ˜20-30 repeats in LPS. However, not all 3000 or more repeat units need be present in the O-Ag capsule used in the compositions described herein, so long as the O-Ag capsule is immunogenic. Preferably, the number of repeats present will be at least 100.

The yih operons (FIG. 1D) are responsible for O-Ag capsule biosynthesis. Mutation of yihO or yihQ yields capsule-negative strains. Immune serum raised to purified O-Ag capsule has only minimal cross-reactivity to intact LPS. Thus, this immunological distinction may be due to the presence of conformational epitopes within the O-Ag capsule that do not exist in LPS. This is important for vaccine development because antibodies directed against LPS tend to be very specific to the S. Enterica serovar that it was isolated from (Hormaeche et al. (1996) Vaccine 14:251-259).

In particular, the genes responsible for O-Ag capsule production in Salmonellae are yihU-yshA, while yihV was thought to encode a kinase, and yihW may encode a transcriptional modifier (FIG. 1D). As detailed below in the experimental section, the inventors herein have confirmed that knocking out transcriptional modifiers of the O-Ag capsule production pathway, in fact increases the production of O-Ag capsule.

In this regard, the function of YihV and YihW on gene expression of the O-Ag capsule operon (yihU-yishA) was confirmed by the inventors herein by recombinantly producing a construct with a deletion of the yihVW gene region. These genes were shown to be repressors of O-Ag capsule production. Thus, the production of mutant constructs with deletions of all or a portion of these genes results in enhanced production of the O-Ag capsule. Methods of knocking out genes are well known in the art and can be used to produce the constructs. One such method is detailed in the examples herein and is described for example, in Datsenko, et al. (2000) Proc. Natl. Acad. Sci 97:6640-6645. However, any known method for deleting all or part of the yihVW region of the operon, can be used in order to enhance production of the O-Ag capsule. By “enhancing O-Ag capsule production” as used herein is meant that production is increased by at least 2 to 1000 times or more as compared to O-Ag capsule production when produced with the entire yihVW gene region present, more preferably 5 to 1000 times or more, such as increased by 2 . . . 5 . . . 10 . . . 20 . . . 30 . . . 40 . . . 50 . . . 75 . . . 100 . . . 150 . . . 200 . . . 300 . . . 400 . . . 500 . . . 750 . . . 1000 . . . times or more, or any integer between these ranges.

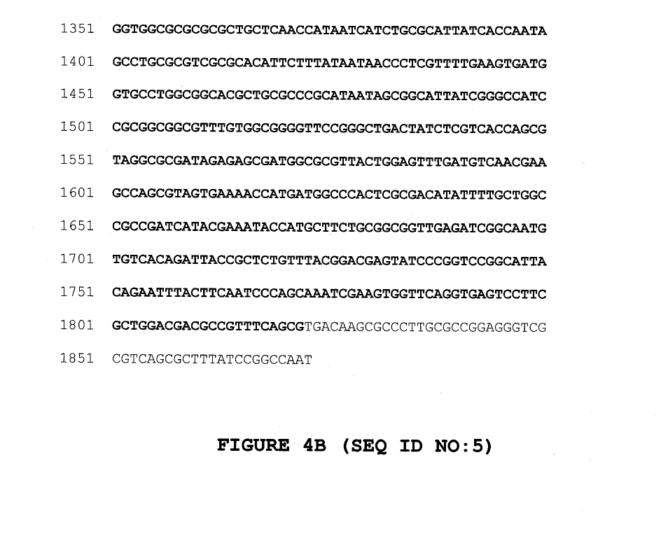

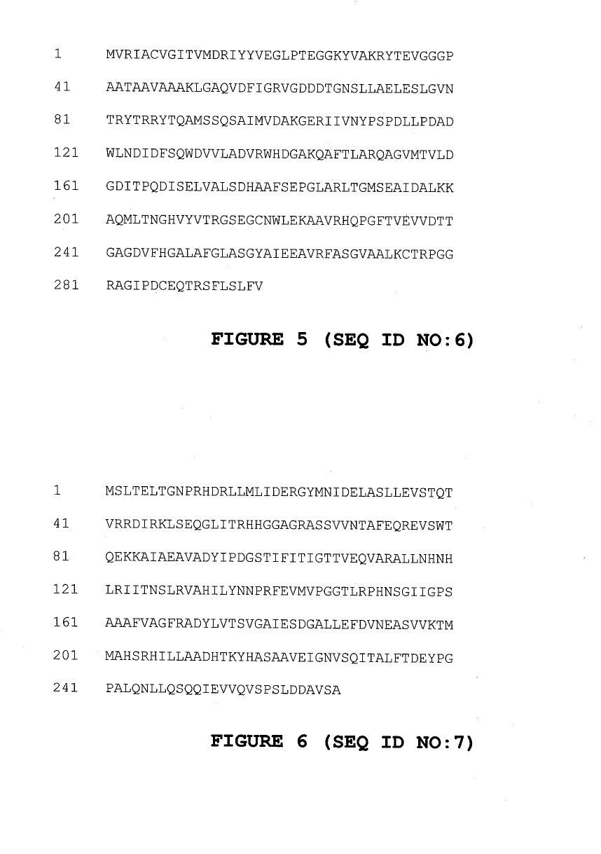

FIGS. 4A-4B (SEQ ID N0:5) show a nucleotide sequence of the yihVW region from a representative S. enterica NTS serovar (S. Typhimurium str. LT2). The coding sequence for yihV begins at nucleotide position 92 and ends at nucleotide position 988. The coding sequence for yih W begins at nucleotide position 1022 and ends at position 1825. The bolded region represents the deletion present in a representative yihVW mutant (corresponding to nucleotide positions 96-1822). The corresponding amino acid sequences for the YihV and YihW proteins encoded by this nucleotide sequence are shown in FIG. 5 (SEQ ID NO:6) and FIG. 6 (SEQ ID N0:7), respectively. It is to be understood that the numbering herein is relative to the reference sequences and that the corresponding positions in the yihVW regions derived from any serovar, type or strain of an S. enterica serovar are also intended to be encompassed by the present invention.

As explained above, deletion of all or a portion of the yihV and/or yihW genes that result in enhanced production of the O-Ag capsule are contemplated herein. Accordingly, all or a portion of YihV may be deleted, and/or all or a portion of YihW may be deleted, so long as the production of the O-Ag capsule is enhanced, as defined above. YihW is predicted to be a DNA-binding protein. Thus, deletion constructs of the invention will typically include at least a deletion of the DNA-binding region of YihW. With reference to FIG. 6 (SEQ ID NO:7), the binding region of YihW is believed to be at approximately positions 8-63. The corresponding positions of the DNA-binding region from other serovars and strains can be readily determined. Deletions of all or a portion of the nucleotide sequence encoding this region are highly desirable. For example, deletions of amino acids 8-63 or more, 20-63 or more, 24-62 or more, or any number within these stated ranges, can be made, so long as the construct retains the ability to enhance O-Ag capsule production as defined above.

Accordingly, the constructs may include all or a portion of yih V, as well as all or a portion of yihW, with all or a portion of the nucleotide sequence encoding the DNA-binding region of YihW deleted. In some embodiments then, only the DNA-binding portion of the nucleotide sequence encoding YihW is deleted, with the remainder of the yihVW region present. Similarly, all or a portion, or none of the yihV gene may be deleted, along with all or a portion of the DNA-binding encoding region of the yihW gene, with all or a portion of the remainder of the yihW gene present. One of skill in the art can readily envision various deletion constructs wherein portions of the yihV and/or yihW genes are eliminated in order to enhance O-Ag capsule production.

The sequences of the genomes of several S. enterica serovars are known, including the yih operons, and can be found at, for example, NCBI Accession nos. CP007804.1; CP001363.1; AE006468.1; NC_016856.1; FQ312003.1; AL513382.1 (all S. Typhimurium); AM933172.1 (S. Enteritidis); CP000026.1; NC_006511.1; CP000857.1 (all S. Paratyphi); AE017220.1; CM001062.1; NZ_CM001062.1 (all S. Choleraesuis); CP00595.1; CP005390.2 (both S. Heidelberg) (all incorporated herein by reference in their entireties). These and other S. enterica sequences can be used to produce mutants which lack all or a portion of the yihV and/or yihW sequences such that production of the O-Ag capsule is enhanced.

### B. Purification of S. Enterica O-Ag Capsule

The immunogenic O-Ag described herein can be purified in any suitable manner (e.g. purification from cell culture, chemical synthesis, etc.) and in various forms (e.g. native, mutant, variants, etc.). Means for preparing such molecules are well understood in the art. The O-Ag capsule is preferably prepared in substantially pure form (i.e. substantially free from other host cell or non host cell proteins).

In addition to recombinant production as described herein, the O-Ag capsule can be conveniently synthesized chemically, by any of several techniques that are known to those skilled in the polysaccharide art. For example, the biosynthetic pathways for S. Enteritidis and S. Typhimurium, as well as other S. Enterica O-Ags have been characterized (see, e.g., Wang et al. (1996) J. Bacteriol. 178:2590-2604; Liu et al. (1995) J. Bacteriol 177:4084-4088; Liu et al. (1993) J. Bacteria 175:33408-3413; McGrath (1991) J Bacteriol. 173:649-654; Fitzgerald et al. (2003) Appl. Environ. Microbiol. 69:6099-6105) and can be used as the basis for synthesis schemes to produce the O-Ag in vitro for use in compositions as detailed herein.

Alternatively, the O-Ags for use in the compositions described herein can be isolated directly from a desired NTS S. enterica serovar. For example, the bacterium can be grown on an appropriate medium, well known in the art. See, e.g., Gibson et al. (2006) J. Bacterial 188:7720-7733. If desired, the bacterium used may be cellulose-deficient in order to facilitate purification. Construction of such S. enterica mutants is well known in the art. See, e.g., White et al. (2003) J Bacteriol. 185:5398-5407. One particularly desirable mutant is derived from Salmonella serovar Typhimurium, and includes a deletion of the gene coding for cellulose synthase and is termed ΔbcsA. See, e.g., White et al. (2003) J Bacteriol. 185:5398-5407 and the examples herein. Also, O-Ag capsule expression can be boosted by over-expressing or under-expressing a modulator of the O-Ag capsule operon or the operon itself. For example, this can be accomplished by deleting all or a portion of yih VW or by over-expressing the entire yih UTSRQPO operon from the Salmonella chromosome and cloning into a suitable plasmid, such as pBR322. The desired NTS S. enterica serovar, such as Serovars Enteritidis (S. Enteritidis), Typhimurium (S. Typhimurium) or Heidelberg (S. Heidelberg), or a cellulose-deficient mutant thereof, can be transformed with the plasmid.

As described above, one convenient method of enhancing expression of the O-Ag capsule is by deleting all or portions of transcriptional modifiers, such as yihV, yihW and/or yihVW.

Cell surface components are then isolated using any of several techniques known in the art, such as but not limited to first producing a crude lysate by disrupting cells using chemical, physical or mechanical means.

Components from the cell membrane can be separated from other cellular molecules e.g., by the use of detergents or organic solvents. Such methods are known to those of skill in the art and are described in, e.g., Protein Purification Applications: A Practical Approach, (E. L. V. Harris and S. Angal, Eds., 1990). One particularly desirable method involves phenol extraction. In this method, cellular debris is pelleted, typically by centrifugation, the supernatant is removed and the cellular debris, which includes the O-ag capsule, is collected for further use (see, e.g., Gibson et al. (2006) J. Bacterid 188:7722-7730).

The O-Ag capsule is then further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like. One of more of these methods can be used in combination and in any order.

For example, one method for further purifying the O-Ag capsule involves affinity purification, such as by immunoaffinity chromatography using specific antibodies. The choice of a suitable affinity resin is within the skill in the art.

Another method of purification employs size-exclusion chromatography, using a bed with an appropriate fraction range for the O-Ag capsule, such as, but not limited to an appropriate SUPEROSE, SUPERDEX, SEPHADEX, SEPHAROSE, SEPHACRYL column packing material (all available from GE Healthcare Life Sciences). Appropriate buffers for use with such columns are well known in the art, including without limitation phosphate-buffered saline (PBS), sodium phosphate, TRIS buffer solutions, and the like at varying pHs, also well within the skill in the art.

Another method of purification involves the use of anion exchange chromatography. This is a particularly useful method as the O-Ag has a lower net charge than LPS and can therefore be separated away from the LPS by anion exchange chromatography. A number of suitable anion exchangers for use with the present invention are known and include without limitation, MACRO PREP Q (strong anion-exchanger available from BioRad, Hercules, Calif.); UNOSPHERE Q (strong anion-exchanger available from BioRad, Hercules, Calf.); POROS 50HQ (strong anion-exchanger available from Applied Biosystems, Foster City, Calf.); POROS 50D (weak anion-exchanger available from Applied Biosystems, Foster City, Calf.); POROS 50PI (weak anion-exchanger available from Applied Biosystems, Foster City, Calf.); SOURCE 30Q (strong anion-exchanger available from Amersham Biosciences, Piscataway, N.J.); DEAE SEPHAROSE (weak anion-exchanger available from Amersham Biosciences, Piscataway, N.J.); Q SEPHAROSE (strong anion-exchanger available from Amersham Biosciences, Piscataway, N.J.).

The anion exchange column is first equilibrated using standard buffers and according to the manufacturer's specifications. Sample is then loaded and two elution buffers can be used, one low salt buffer and one high salt buffer. Fractions are collected following each of the low salt and high salt washes and the desired material is detected in the fractions using standard techniques, such as monitoring UV absorption at, e.g., 620 nm, Western blot, and the like.

Appropriate buffers for use with the anion exchange columns are well known in the art and are generally cationic or zwitterionic in nature. Such buffers include, without limitation, buffers with the following buffer ions: N-methylpiperazine; piperazine; Bis-Tris; Bis-Tris propane; Triethanolamine; Tris; N-methyldiethanolamine; 1,3-diaminopropane; ethanolamine; acetic acid, and the like. To elute the sample, the ionic strength of the starting buffer is increased using a salt, such as NaC1, KC1, sulfate, formate or sodium acetate (NaOAc), at an appropriate pH.

In one embodiment of the invention, the anion exchange column is first treated with a low salt concentration, e.g., 10-100 mM of an NaOAc or NaCl buffer, such as 10 . . . 15 . . . 20 . . . 25 . . . 30 . . . 35 . . . 40 . . . 45 . . . 50 . . . 55 . . . 60 . . . 65 . . . 100 mM, or any concentration within these ranges. Following initial treatment, the column is then treated with a higher salt concentration or with another buffer with a greater ionic strength. One example for use as the second buffer is an NaOAc buffer or a Tris-based buffer with a concentration of 0.05-5 M, preferably 0.5- 3 M, such as 0.5 . . . 0.75 . . . 1 . . . 1.25 . . . 1.5 . . . 2 . . . 2.5 . . . 3 M, or any concentration within these stated ranges.

In some embodiments, more than one of the above methods can be used in combination, such as anion exchange chromatography in combination with size-exclusion chromatography. If two or more column types are used, they can be used in any order. For example, a size-exclusion column can be used first, followed by an anion exchange column, or vise versa.

For example, during anion exchange chromatography, both LPS and O-Antigen capsule may be eluted together, depending on the conditions. However, fractions eluted with less salt will typically have more LPS than those eluted with higher salt concentrations. Fractions containing the O-Antigen capsule can be determined using Western blots. Size exclusion chromatography can then be used to separate most of the LPS away from the O-Antigen capsule. The peak corresponding to the O-Antigen capsule can be determined after testing each fraction from the size exclusion column on a Western blot.

The O-Ag can also be further purified to remove the LPS using conventional techniques well known in the art, such as by phase separation, using a suitable detergent. See, e.g., Adam et al. (1995) Analytic. Biochem. 225:321-327. Examples of detergents suitable for use include decanoyl-N-methylglucamide, diethylene glycol monopentyl-ether, n-dodecyl β-D-glucopyranoside, ethylene oxide condensates of fatty alcohols (e.g., sold under the trade name LUBROL), polyoxyethylene ethers of fatty acids (particularly C₁₂ -C₂₀ fatty acids), polyoxyethylene sorbitan fatty acid ethers (e.g., sold under the trade name TWEEN), sorbitan fatty acid ethers (e.g., sold under the trade name SPAN), a polyethylene glycol detergent (e.g., sold under the trade name TRITON X-114), a polyethylene oxide detergent (e.g., sold under the trade name TRITON X-100), octylphenoxypolyethoxyethanol (e.g., sold under the trade name NONIDET P-40).

Once purification is completed, the amount of LPS remaining in the product can be tested using the Limulus Amebocyte Lysate (LAL) Chromogenic Endotoxin Quantitation Kit (Pierce) and approved for use by the FDA. Endotoxin is measured in Endotoxin Units per milliliter (EU/mL). One EU equals approximately 0.1 to 0.2 ng endotoxin/mL of solution. Due to the serious risks associated with endotoxin contamination, the FDA has set limits on concentration of endotoxin for medical devices and parenteral drugs. Currently there are three forms of the LAL assay, each with different sensitivities, any of which can be used for purposes of the present invention. The LAL gel clot assay can detect down to 0.03 EU/mL while the LAL kinetic turbidimetric and chromogenic assays can detect down to 0.01 EU/mL.

The effects of endotoxin are related to the amount of endotoxin in the product dose administered to a patient. Because the dose varies from product to product, the endotoxin limit is expressed as K/M. K is 5.0 EU/kilogram (kg), which represents the approximate threshold pyrogen dose for humans and rabbits. That is the level at which a product is adjudged pyrogenic or non-pyrogenic. M represents the rabbit pyrogen test dose or the maximum human dose per kilogram that would be administered in a single one hour period, whichever is larger. For example, a non-intrathecal drug product that has a maximum human dose of 10 ml/kg. Thus, Endotoxin limit=K 5 EU/kg= - - - =0.5 EU/ml M 10 ml/kg.

Preferably then, and depending on whether the O-Ag preparation is intended for human or animal use, the amount of LPS remaining in the final product will typically be under 2×10⁶ EU/mg, such as under 2×10⁵ EU/mg, 2×10⁴ EU/mg, 5 x 10³ EU/mg, 4 x 10³ EU/mg, 3×10³ EU/mg, 2.5×10³ EU/mg, or any number between these ranges, where one EU=0.1 ng of E. coli LPS/mL of solution.

### C. Antibodies

The purified O-Ag of the present invention can be used to produce antibodies for therapeutic (e.g., passive immunization), diagnostic and purification purposes. These antibodies may be polyclonal or monoclonal antibody preparations, monospecific antisera, human antibodies, or may be hybrid or chimeric antibodies, such as humanized antibodies, altered antibodies, F(ab′)₂ fragments, F(ab) fragments, Fv fragments, single-domain antibodies, dimeric or trimeric antibody fragment constructs, minibodies, or functional fragments thereof which bind to the antigen in question. Antibodies are produced using techniques well known to those of skill in the art and disclosed in, for example, U.S. Pat. Nos. 4,011,308; 4,722,890; 4,016,043; 3,876,504; 3,770,380; and 4,372,745.

For example, the O-Ags can be used to produce specific polyclonal and monoclonal antibodies for use in diagnostic and detection assays, for purification and for use as therapeutics, such as for passive immunization. Such polyclonal and monoclonal antibodies specifically bind to the O-Ag in question. In particular, the O-Ags can be used to produce polyclonal antibodies by administering the antigen to a mammal, such as a mouse, a rat, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Mouse and/or rabbit monoclonal antibodies directed against epitopes present in the O-Ags can also be readily produced. In order to produce such monoclonal antibodies, the mammal of interest, such as a rabbit or mouse, is immunized, such as by mixing or emulsifying the antigen in saline, preferably in an adjuvant such as Freund's complete adjuvant (“FCA”), and injecting the mixture or emulsion parenterally (generally subcutaneously or intramuscularly). The animal is generally boosted 2-6 weeks later with one or more injections of the antigen in saline, preferably using Freund's incomplete adjuvant (“FIA”).

Antibodies may also be generated by in vitro immunization, using methods known in the art. See, e.g., James et al., J. Immunol. Meth. (1987) 100:5-40.

Polyclonal antisera is then obtained from the immunized animal. However, rather than bleeding the animal to extract serum, the spleen (and optionally several large lymph nodes) is removed and dissociated into single cells. If desired, the spleen cells (splenocytes) may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated splenocytes, are then induced to fuse with cells from an immortalized cell line (also termed a “fusion partner”), to form hybridomas. Typically, the fusion partner includes a property that allows selection of the resulting hybridomas using specific media. For example, fusion partners can be hypoxanthine/aminopterin/thymidine (HAT)-sensitive.

If rabbit-rabbit hybridomas are desired, the immortalized cell line will be from a rabbit. Such rabbit-derived fusion partners are known in the art and include, for example, cells of lymphoid origin, such as cells from a rabbit plasmacytoma as described in Spieker-Polet et al., Proc. Natl. Acad. Sci. USA (1995) 92:9348-9352 and U.S. Pat. No. 5,675,063, or the TP-3 fusion partner described in U.S. Pat. No. 4,859,595, incorporated herein by reference in their entireties. If a rabbit-mouse hybridoma or a rat-mouse or mouse-mouse hybridoma, or the like, is desired, the mouse fusion partner will be derived from an immortalized cell line from a mouse, such as a cell of lymphoid origin, typically from a mouse myeloma cell line. A number of such cell lines are known in the art and are available from the ATCC.

Fusion is accomplished using techniques well known in the art. Chemicals that promote fusion are commonly referred to as fusogens. These agents are extremely hydrophilic and facilitate membrane contact. One particularly preferred method of cell fusion uses polyethylene glycol (PEG). Another method of cell fusion is electrofusion. In this method, cells are exposed to a predetermined electrical discharge that alters the cell membrane potential. Additional methods for cell fusion include bridged-fusion methods. In this method, the antigen is biotinylated and the fusion partner is avidinylated. When the cells are added together, an antigen-reactive B cell-antigen-biotin-avidin-fusion partner bridge is formed. This permits the specific fusion of an antigen-reactive cell with an immortalizing cell. The method may additionally employ chemical or electrical means to facilitate cell fusion.

Following fusion, the cells are cultured in a selective medium (e.g., HAT medium). In order to enhance antibody secretion, an agent that has secretory stimulating effects can optionally be used, such as IL-6. See, e.g., Liguori et al., Hybridoma (2001) 20:189-198. The resulting hybridomas can be plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice). For example, hybridomas producing S. enterica NTS O-Ag-specific antibodies can be identified using RIA or ELISA and isolated by cloning in semi-solid agar or by limiting dilution. Clones producing the desired antibodies can be isolated by another round of screening.

An alternative technique for generating the monoclonal antibodies is the selected lymphocyte antibody method (SLAM). This method involves identifying a single lymphocyte that is producing an antibody with the desired specificity or function within a large population of lymphoid cells. The genetic information that encodes the specificity of the antibody (i.e., the immunoglobulin V_{H} and V_{L} DNA) is then rescued and cloned. See, e.g., Babcook et al., Proc. Natl. Acad. Sci. USA (1996) 93:7843-7848, for a description of this method.

For further descriptions of rabbit monoclonal antibodies and methods of making the same from rabbit-rabbit and rabbit-mouse fusions, see, e.g., U.S. Pat. No. 5,675,063 (rabbit-rabbit); U.S. Pat. No. 4,859,595 (rabbit-rabbit); U.S. Pat. No. 5,472,868 (rabbit-mouse); and U.S. Pat. No. 4,977,081 (rabbit-mouse). For a description of the production of conventional mouse monoclonal antibodies, see, e.g., Kohler and Milstein, Nature (1975) 256:495-497.

It may be desirable to provide chimeric antibodies. By “chimeric antibodies” is intended antibodies that are preferably derived using recombinant techniques and which comprise both human (including immunologically “related” species, e.g., chimpanzee) and non-human components. Such antibodies are also termed “humanized antibodies.” Preferably, humanized antibodies contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human inununoglobulin. For further details see Jones et al., Nature (1986) 331:522-525; Riechmann et al., Nature (1988) 332:323-329; and Presta, Curr. Op. Struct. Biol. (1992) 2:593-596.

Also encompassed are xenogeneic or modified antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598.

Antibody fragments which retain the ability to recognize the antigen of interest, will also find use herein. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)2 fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as FV. See, e.g., Inbar et al., Proc. Nat. Acad. Sci. USA (1972) 69:2659-2662; Hochman et al., Biochem. (1976) 15:2706-2710; and Ehrlich et al., Biochem. (1980) 19:4091-4096.

A phage-display system can be used to expand antibody molecule populations in vitro. Saiki, et al., Nature (1986) 324:163; Scharf et al., Science (1986) 233:1076; U.S. Pat. Nos. 4,683,195 and 4,683,202; Yang et al., J Mol Biol. (1995) 254:392; Barbas, HI et al., Methods: Comp. Meth Enzymol. (1995) 8:94; Barbas, III et al., Proc Natl Acad Sci USA (1991) 88:7978.

Once generated, the phage display library can be used to improve the immunological binding affinity of the Fab molecules using known techniques. See, e.g., Figini et al., J. Mol. Biol. (1994) 239:68. The coding sequences for the heavy and light chain portions of the Fab molecules selected from the phage display library can be isolated or synthesized, and cloned into any suitable vector or replicon for expression. Any suitable expression system can be used, including those described above.

Single chain antibodies can also be produced. A single-chain Fv (“sFv” or “scFv”) polypeptide is a covalently linked VH-VL heterodimer which is expressed from a gene fusion including VH- and VL-encoding genes linked by a peptide-encoding linker. Huston et al., Proc. Nat. Acad. Sci. USA (1988) 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al., Proc. Nat. Acad. Sci. USA (1988) 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Patent Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present invention. Minibodies are sFv polypeptide chains which include oligomerization domains at their C-termini, separated from the sFv by a hinge region. Pack et al., Biochem. (1992)3_1:1579-1584. 1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al., Biochem. (1992) 31:1579-1584; Cumber et al., J. Immunology (1992) 149B:120-126.

Polynucleotide sequences encoding the antibodies and immunoreactive fragments thereof, described above, are readily obtained using standard techniques, well known in the art.

For subjects known to have an S. enterica NTS-related disease, an anti-S. enterica NTS O-Agantibody may have therapeutic benefit and can be used to confer passive immunity to the subject in question. Alternatively, antibodies can be used in diagnostic applications, described further below, as well as for purification of the antigen of interest.

### D. Compositions

The S. enterica NTS O-Ag capsule or antibodies, can be formulated into compositions for delivery to subjects for either inhibiting infection, or for enhancing an immune response to the antigen. Moreover, the compositions can be used to effect a reduction of the amount of S. enterica in the intestinal tract in the subject, thus reducing transmission of disease by reducing the amount of fecal shedding of bacteria.

Compositions of the invention may comprise or be co-administered with non-S. enterica NTS O-Ag capsules or with a combination of S. enterica antigens. Methods of preparing such formulations are described in, e.g., Remington's Pharmaceutical Sciences, Mack Publishing Company, Easton, Pennsylvania, 18 Edition, 1990. The compositions of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the composition may also be added to the formulation. Such adjuvants include any compound or compounds that act to increase an immune response to an O-Ag capsule or combination of antigens, thus reducing the quantity of antigen necessary in the vaccine, and/or the frequency of injection necessary in order to generate an adequate immune response. Adjuvants may include for example, muramyl dipeptides, AVRIDINE, aluminum hydroxide, dimethyldioctadecyl ammonium bromide (DDA), oils, oil-in-water emulsions, water-in-oil emulsions, such as described in U.S. Pat. No. 7,279,163, incorporated herein by reference in its entirety, saponins, cytokines, and other substances known in the art.

Thus, for example, adjuvants may include for example, emulsifiers, muramyl dipeptides, avridine, aqueous adjuvants such as aluminum hydroxide, chitosan-based adjuvants, and any of the various saponins, oils, and other substances known in the art, such as AMPHIGEN™ which comprises de-oiled lecithin dissolved in an oil, usually light liquid paraffin. In vaccine preparations AMPHIGEN™ is dispersed in an aqueous solution or suspension of the immunizing antigen as an oil-in-water emulsion. Other adjuvants are LPS, bacterial cell wall extracts, bacterial DNA, synthetic oligonucleotides and combinations thereof (Schijns et al., Curr. Opi. Immunol. (2000) 12:456), Mycobacterial phlei (M phlei) cell wall extract (MCWE) (U.S. Pat. No. 4,744,984), M phlei DNA (M-DNA), M-DNA-M phlei cell wall complex (MCC). For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. See, e.g., Remington's Pharmaceutical Sciences, supra. A particularly preferred oil component is the oil-in-water emulsion sold under the trade name of EMULSIGEN PLUS™, comprising a light mineral oil as well as 0.05% formalin, and 30 μg/mL gentamicin as preservatives), available from MVP Laboratories, Ralston, NE. Also of use herein is an adjuvant known as “VSA3” which is a modified form of EMULSIGEN PLUS™ which includes DDA (see, U.S. Pat. No. 5,951,988, incorporated herein by reference in its entirety). Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369 386). Specific compounds include dimethyldioctadecylammonium bromide (DDA) (available from Kodak) and N,N-dioctadecyl-N,N-bis(2-hydroxyethyl)propanediamine (“AVRIDINE”). The use of DDA as an immunologic adjuvant has been described; see, e.g., the Kodak Laboratory Chemicals Bulletin 56(1):1 5 (1986); Adv. Drug Deliv. Rev. 5(3):163 187 (1990); 1 Controlled Release 7:123 132 (1988); Clin. Exp. Immunol. 78(2):256 262 (1989); J. Immunol. Methods 97(2):159 164 (1987); Immunology 58(2):245 250 (1986); and Int. Arch. Allergy Appl. Immunol. 68(3):201 208 (1982). AVRIDINE is also a well-known adjuvant. See, e.g., U.S. Pat. No. 4,310,550, incorporated herein by reference in its entirety, which describes the use of N,N-higher alkyl-N,N-bis(2-hydroxyethyl)propane diamines in general, and AVRIDINE in particular, as vaccine adjuvants. U.S. Pat. No. 5,151,267 to Babiuk, incorporarted herein by reference in its entirety, and Babiuk et al. (1986) Virology 159:57 66, also relate to the use of AVRIDINE as a vaccine adjuvant.

Moreover, the O-Ag capsule may be conjugated to a carrier protein in order to enhance the immunogenicity thereof. The conjugation of polysaccharides to carrier proteins is well known and reviewed, e.g., in Lindberg (1999) Vaccine 17 Suppl 2:S28-36; Buttery et al. (2000) J. R. Coll. Physicians Lond 34:163-168; Ahmad et al. (1999) Infect. Dis. Clin. North Am. 13:113-133, vii; Goldblatt (1998) J. Med. Microbiol. 47:563-567; EP 0 477 508; U.S. Pat. No. 5,306,492; PCT Publ. WO 98/42721; Dick et al. in Conjugate Vaccines (eds. Cruse et al.) Karger, Basel 1989, Vol. 10, 48-114; Hermanson (1996) Bioconjugate Techniques, Academic Press, San Diego, all incorporated herein by reference in their entireties.

Suitable proteins include bacterial toxins that are immunologically effective carriers that have been rendered safe by chemical or genetic means for administration to a subject. Examples include inactivated bacterial toxins such as diphtheria toxoid, CRM₁₉₇, tetanus toxoid, pertussis toxoid, E. coli LT, E. coli ST, and exotoxin A from Pseudomonas aeruginosa. Bacterial outer membrane proteins such as, outer membrane complex c (OMPC), porins, transferrin binding proteins, pneumolysis, pneumococcal surface protein A (PspA), pneumococcal adhesin protein (PsaA), or pneumococcal surface proteins BVH-3 and BVH-11 can also be used. Other proteins, such as protective antigen (PA) of Bacillus anthracia, ovalbumin, keyhole limpet hemocyanin (KLH), human serum albumin, bovine serum albumin (BSA) and purified protein derivative of tuberculin (PPD) can also be used. The proteins are preferably proteins that are non-toxic and non-reactogenic and obtainable in sufficient amount and purity that are amenable to the conjugation methods of preferred embodiments. For example, diphtheria toxin can be purified from cultures of Corynebacteria diphtheriae and chemically detoxified using formaldehyde to yield a suitable protein.

Fragments of the native toxins or toxoids, which contain at least one T-cell epitope, are also useful, as are outer membrane protein complexes, as well as certain analogs, fragments, and/or analog fragments of the various proteins listed above. The proteins can be obtained from natural sources, can be produced by recombinant technology, or by synthetic methods as are known in the art. Analogs can be obtained by various means, for example, certain amino acids can be substituted for other amino acids in a protein without appreciable loss of interactive binding capacity with structures such as, for example, antigen-binding regions of antibodies or binding sites on substrate molecules. Other proteins can also be employed, such as those containing surface exposed glutamic acid or aspartic acid groups.

The carrier molecule may be covalently conjugated to the O-Ag capsule directly or via a linker. Any suitable conjugation reaction can be used, with any suitable linker where desired. There are many conjugation reactions that have been employed for covalently linking polysaccharides to proteins. Three of the more commonly employed methods include: 1) reductive amination, wherein the aldehyde or ketone group on one component of the reaction reacts with the amino or hydrazide group on the other component, and the C═N double bond formed is subsequently reduced to C—N single bond by a reducing agent; 2) cyanylation conjugation, wherein the polysaccharide is activated either by cyanogens bromide

(CNBr) or by 1-cyano-4-dimethylammoniumpyridinium tetrafluoroborate (CDAP) to introduce a cyanate group to the hydroxyl group, which forms a covalent bond to the amino or hydrazide group upon addition of the protein component; and 3) a carbodiimide reaction, wherein carbodiimide activates the carboxyl group on one component of the conjugation reaction, and the activated carbonyl group reacts with the amino or hydrazide group on the other component. These reactions are also frequently employed to activate the components of the conjugate prior to the conjugation reaction. U.S. Pat. No. 8,465,749, incorporated herein by reference in its entirety, describes these and additional methods for preparing polysaccharide/protein conjugates for use as vaccines.

Once prepared, the O-Ag capsule formulations will contain a “pharmaceutically effective amount” of the active ingredient, that is, an amount capable of achieving the desired response in a subject to which the composition is administered. In the treatment and prevention of NTS disease, for example, a “pharmaceutically effective amount” would preferably be an amount which reduces or ameliorates the symptoms of the disease in question. Additionally, prevention or treatment in the context of the present invention can be a reduction of the amount of S. enterica NTS in the intestinal tract, thus reducing transmission of disease by reducing the amount of fecal shedding of bacteria. Thus, an asymptomatic subject is still considered to have been “treated” if the amount of S. enterica NTS in the intestinal tract is reduced.

The exact amount is readily determined by one skilled in the art using standard tests. The active ingredient will typically range from about 1% to about 95% (w/w) of the composition, or even higher or lower if appropriate. With the present formulations, 1 μg to 2 mg, such as 100 μg to 1 mg, of active ingredient per ml of injected solution should be adequate to treat or prevent infection when a dose of 1 to 5 ml per subject is administered. The quantity to be administered depends on the subject to be treated, the capacity of the subject's immune system to synthesize antibodies, and the degree of protection desired. Effective dosages can be readily established by one of ordinary skill in the art through routine trials establishing dose response curves.

The composition can be administered parenterally, e.g., by intratracheal, intramuscular, subcutaneous, intraperitoneal, intravenous injection, or by delivery directly to the lungs, such as through aerosol administration. The subject is administered at least one dose of the composition. Moreover, the subject may be administered as many doses as is required to bring about the desired biological effect.

Additional formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject antigens by the nasal mucosa.

Controlled or sustained release formulations are made by incorporating the antigen into carriers or vehicles such as liposomes, nonresorbable impermeable polymers such as ethylenevinyl acetate copolymers and HYTREL copolymers, swellable polymers such as hydrogels, resorbable polymers such as collagen and certain polyacids or polyesters such as those used to make resorbable sutures, polyphosphazenes, alginate, microparticles, gelatin nanospheres, chitosan nanoparticles, and the like. The antigens described herein can also be delivered using implanted mini-pumps, well known in the art.

Prime-boost methods can be employed where one or more compositions are delivered in a “priming” step and, subsequently, one or more compositions are delivered in a “boosting” step. In certain embodiments, priming and boosting with one or more compositions described herein is followed by additional boosting. The compositions delivered can include the same antigens, or different antigens, given in any order and via any administration route.

### E. Tests to Determine the Efficacy of an Immune Response

One way of assessing efficacy of therapeutic treatment involves monitoring infection after administration of a composition of the invention. One way of assessing efficacy of prophylactic treatment involves monitoring immune responses against the S. enterica NTS O-Ag capsule in the compositions of the invention after administration of the composition. Moreover, efficacy of the compositions can be determined by assessing whether a reduction of the amount of S. enterica in the intestinal tract in the subject is achieved, thus reducing transmission of disease by reducing the amount of fecal shedding of bacteria.

Another way of assessing the immunogenicity of the O-Ag capsule component of the immunogenic compositions of the present invention is to screen the subject's sera by immunoblot. A positive reaction indicates that the subject has previously mounted an immune response to the O-Ag component, that is, the O-Ag is an immunogen. This method may also be used to identify epitopes.

Another way of checking efficacy of therapeutic treatment involves monitoring infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the antigens in the compositions of the invention after administration of the composition. Typically, serum-specific antibody responses are determined post-immunization but pre-challenge whereas mucosal specific antibody body responses are determined post-immunization and post-challenge. The immunogenic compositions of the present invention can be evaluated in in vitro and in vivo animal models prior to host administration.

The efficacy of immunogenic compositions of the invention can also be determined in vivo by challenging animal models of infection with the immunogenic compositions. The immunogenic compositions may or may not be derived from the same strains as the challenge strains. Preferably the immunogenic compositions are derivable from the same strains as the challenge strains.

The immune response may be one or both of a TH1 immune response and a TH2 response. The immune response may be an improved or an enhanced or an altered immune response. The immune response may be one or both of a systemic and a mucosal immune response. Preferably the immune response is an enhanced systemic and/or mucosal response.

An enhanced systemic and/or mucosal immunity is reflected in an enhanced TH I and/or TH2 immune response. Preferably, the enhanced immune response includes an increase in the production of IgG1 and/or IgG2a and/or IgA. Preferably the mucosal immune response is a TH2 immune response. Preferably, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG 1 production.

A TH1 immune response may include one or more of an increase in CTLs, an increase in one or more of the cytokines associated with a TH1 immune response (such as IL-2, IFNy, and TNFI3), an increase in activated macrophages, an increase in NK activity, or an increase in the production of IgG2a. Preferably, the enhanced TH1 immune response will include an increase in IgG2a production.

The immunogenic compositions of the invention will preferably induce long lasting (e.g., neutralizing) antibodies and a cell mediated immunity that can quickly respond upon exposure to one or more infectious antigens. By way of example, evidence of neutralizing antibodies in blood samples from the subject is considered as a surrogate parameter for protection.

### F. Diagnostic Assays

As explained above, the S. enterica NTS O-Ag capsules, variants and immunogenic fragments thereof, may also be used as diagnostics to detect the presence of reactive antibodies of S. enterica NTS, in a biological sample in order to determine the presence of infection. Conversely, antibodies as described herein can be used to detect the presence of S. enterica NTS in a biological sample. For example, the presence of reactive antibodies and antigens can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound antibody or antigen in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. Typically, a solid support is first reacted with a solid phase component (e.g., one or more S. enterica NTS O-Ag capsules or antibodies) under suitable binding conditions such that the component is sufficiently immobilized to the support. Sometimes, immobilization of the antigen or antibody to the support can be enhanced by first coupling to a protein with better binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigens and/or antibodies to the support include polysaccharides, polylactic acids, polyglycolic acids, polymeric amino acids, amino acid copolymers, and the like. Such molecules and methods of coupling these molecules to the antigens, are well known to those of ordinary skill in the art. See, e.g., Brinkley, M. A. Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing ligand moieties (e.g., antibodies toward the immobilized antigens or antigens that bind the antibodies) under suitable binding conditions. After washing to remove any non-bound ligand, a secondary binder moiety is added under suitable binding conditions, wherein the secondary binder is capable of associating selectively with the bound ligand. The presence of the secondary binder can then be detected using techniques well known in the art.

More particularly, an ELISA method can be used, wherein the wells of a microtiter plate are coated with an S. enterica NTS O-Ag capsule. A biological sample containing or suspected of containing anti-S. enterica immunoglobulin molecules is then added to the coated wells. After a period of incubation sufficient to allow antibody binding to the immobilized antigen, the plate(s) can be washed to remove unbound moieties and a detectably labeled secondary binding molecule added. The secondary binding molecule is allowed to react with any captured sample antibodies, the plate washed and the presence of the secondary binding molecule detected using methods well known in the art.

Thus, in one particular embodiment, the presence of bound anti-S. enterica ligands from a biological sample can be readily detected using a secondary binder comprising an antibody directed against the antibody ligands. A number of immunoglobulin (Ig) molecules are known in the art which can be readily conjugated to a detectable enzyme label, such as horseradish peroxidase, alkaline phosphatase or urease, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the S. enterica NTS O-Ag capsules and antibodies specific therefor form complexes under precipitating conditions. In one particular embodiment, S. enterica NTS O-Ag capsules can be attached to a solid phase particle (e.g., an agarose bead or the like) using coupling techniques known in the art, such as by direct chemical or indirect coupling. The antigen-coated particle is then contacted under suitable binding conditions with a biological sample suspected of containing antibodies for the S. enterica NTS O-Ag capsules. Cross-linking between bound antibodies causes the formation of particle-antigen-antibody complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

In yet a further embodiment, an immunoaffinity matrix can be provided, wherein a polyclonal population of antibodies from a biological sample suspected of containing anti-S. enterica NTS O-Ag capsules is immobilized to a substrate. In this regard, an initial affinity purification of the sample can be carried out using immobilized antigens. The resultant sample preparation will thus only contain anti-S. enterica NTS moieties, avoiding potential nonspecific binding properties in the affinity support. A number of methods of immobilizing immunoglobulins (either intact or in specific fragments) at high yield and good retention of antigen binding activity are known in the art. Not being limited by any particular method, immobilized protein A or protein G can be used to immobilize immunoglobulins.

Accordingly, once the immunoglobulin molecules have been immobilized to provide an immunoaffinity matrix, labeled S. enterica NTS O-Ag capsules are contacted with the bound antibodies under suitable binding conditions. After any non-specifically bound antigen has been washed from the immunoaffinity support, the presence of bound antigen can be determined by assaying for label using methods known in the art.

Additionally, antibodies raised to the S. enterica NTS O-Ag capsules, rather than the antigens themselves, can be used in the above-described assays in order to detect the presence of antibodies thereto in a given sample. These assays are performed essentially as described above and are well known to those of skill in the art.

### G. Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a third component comprising an adjuvant.

The kit can also comprise a package insert containing written instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions of the invention.

Similarly, antibodies can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

### 3. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Bacterial Strains and Growth Conditions

The bacterial strains used in the examples were Salmonella ser. Typhimurium (ATCC 14028s) and Salmonella ser. Enteridis 27655-3b (Feutrier et al. (1986) J. Bacteriol. 168:221-227). Cellulose-deficient mutants were generated (see, White et al. (2003) J. Bacteriol. 185:5398-5407; and Zogaj et al. (2001) Mol. Microbiol. 39:1452-1463) in order to facilitate purification of the O-Ag capsule. These mutants, termed ΔbcsA mutants, included an in-frame deletion of 1998 by in bcsA (encoding amino acids 165 to 828 in BcsA). Briefly, primers as detailed in White et al. (2003) J. Bacteriol. 185:5398-5407, were generated and used for PCR in S. Typhimurium 14028 genomic DNA. Restriction digest was used on the PCR product with EcoRI-PstI to generate fragment #1 and PstI-HindIII to generate fragment #2. These fragments were sequentially cloned into EcoRI-HindIII-cut pTZ18R (Amersham Biosciences) and subcloned into pHSG415 (Hashimoto-Gotoh et al. (1981) Gene 16:227-235) and electroporated into S. Typhimurium 14028-3b. Strains that were ampicillin-resistant were selected by growing at 42° C. with ampicillin and positive mutant clones selected.

Bacteria were gown at 28° C. for 5 days on agar plates supplemented with 0.05% yeast extract, 1% glucose, 10 mM sodium phosphate dibasic (Na₂HPO₄), 0.1% ammonium chloride (NH₄C1) and 0.3% potassium phosphate monobasic (KH₂PO₄). Cellular material was then collected and stored at 4° C. for future use.

### Deletion of yihVW from the Chromosome

Competent cells of S. Typhimurium 14028 containing pKD46, which is used for lambda-red based chromosomal insertion, were generated. PCR products containing the cat gene, which codes for chloramphenicol resistance, flanked by 50 by regions corresponding to the beginning ofyihV and the end ofyihW were generated from pKD3 (Datsenko et al. (2000) Proc. Natl. Acad. Sci. 97:6640-6645) using the following primers:

yihVWkoFOR:
(SEQ ID NO: 1)
(TTCGTGAAATTAAAATGAGCACATCGAAAATGCTTGAGGAATGACCA
TGGGTGTAGGCTGGAGCTGCTTC);
and
yihVWkoREV:
(SEQ ID NO: 2)
(TTGGCCGGATAAAGCGCTGACGCGACCCTCCGGCGCAAGGGCGCTTG
TCACCTCCTTAGTTCCTATTCCG).

Cells were plated on 9 μg/mL chloramphenicol plates and incubated overnight at 37° C. Positive clones were re-streaked on to 30 μg/mL chloramphenicol plates and incubated overnight at 37° C. Positive clones were checked with PCR for the deletion of yihVW using the following primers:

Forward: detect1:
(SEQ ID NO: 3)
(GCACATCGAAAATGCTTGAGGA);
Reverse: detect 2
(SEQ ID NO: 4)
(ATATCGCCTGCATCACAGCG).

### P22 Phage Transduction

P22 phage was used to move the mutation into a clean S. Typhimurium background. This avoids the possibility of any secondary genetic mutations generated as part of the lambda-red recombination protocol. The P22 phage was used to move the yihVW::cat fragment from S. Typhimurium 14028 ΔyihVW into the S. Typhimurium 14028 ΔbcsA strain, following standard procedures (Maloy, et al. Genetic analysis ofpathogenic bacteria: a laboratory manual. (Cold Spring Harbor Laboratory Press, 1996). After the final ΔyihVW::cat strains were generated, the cat gene was deleted to generate chloramphenicol-sensitive strains. To delete the cat gene, pCP20 was electroporated into the S. Typhimurium 14028 ΔyihVW::cm strain, and plated on ampicillin agar and incubated overnight at 37° C. A single positive colony was serially diluted, and dilutions 10⁻⁵ and 10⁻⁶ were plated on ampicillin plates and incubated at 42° C. to cure the cells of the pCP20. Loss of chloramphenicol and ampicillin markers was confirmed by plating on LB, chloramphenicol and ampicillin media. Cells that grew only on LB were selected for further screening.

### EXAMPLE 1

### Purification of O-Ag Capsule

Cell surface components were first purified by scraping the agar surfaces and resuspending in 1% phenol, then centrifuging at 11500 rpm at 4° C. for 4 hours. The pellet was discarded and 4 volumes of ice cold acetone was added to the supernatant, while stirring. Precipitation was allowed to occur overnight at −20° C. after which the precipitate was centrifuged at 3200 rpm at 4° C. for 15 minutes and dried overnight. The dried precipitate was then dissolved in dH₂O and dialyzed while stirring for 24 hours at 4° C., frozen at -80° C. for at least an hour, then lyophilized. The lyophilized precipitate was stored at 4° C. for future use.

Cell surface components were further purified using anion exchange and size exclusion chromatography as follows. Buffers used in the anion exchange chromatography were:
A: 15mM NaOAc, 0.05% Triton X-100 pH 5.5
B: 1.5M NaOAc, 0.05% Triton X-100, pH 5.5
C: 100mM NaOAc, 0.05% Triton X-100, pH 5.5
D: 2M NaCl

The lyophilyzed O-Antigen capsule was dissolved with 95 ml of Buffer A and 0.1 ml of 10% sodium azide was added. The dissolved sample was placed in a 37° C. water bath for 10-15 minutes. The sample (pH 5.53) was sterile filtered through a 0.22 um filter. 100 ml of the sample was loaded onto a Q SEPHAROSE FF xk50/11.5 column. A flow rate of 8.5 ml/min was used. The column was washed with 100% of Buffer A for 2.0 column volumes. The gradient was started and 7% of Buffer B was added and held for 1.6 column volumes. Buffer B was increased to 17% and held for 1.25 column volumes followed by increasing Buffer B to 50% and holding for 1.25 column volumes. Finally, Buffer B was increased to 100% and held for 1.5 column volumes. The resin was flushed with 2.0 column volumes of Buffer D.

The cleanest part of the 0-Antigen capsule eluted with 17% Buffer B and less pure parts eluted with 7% Buffer B. The peaks that eluted with 7% Buffer B were kept separate and run separately on size exclusion resin. Western blot confirmed the location of the 0-Antigen capsule on the anion exchange fractions.

The buffer used for size exclusion chromatography was 50 mM NH₄HCO₃, pH 7.75. Fractions from the anion exchange chromatography were pooled according to purity and concentrated using a 50K concentrator. The sample was sterile filtered through a 0.22 μm syringe tip filter and loaded onto a SUPERDEX 5300 prep grade xk26/95 column. A flow rate of 0.45 ml/min was used. The length of elution was 3 column volumes, however the O-Antigen capsule was off the resin in 1 column volume. Western blot was used to confirm the location of the 0-Antigen capsule in the anion exchange fractions.

Following chromatography, polysaccharide (O-Ag capsule)-containing fractions were pooled and dialyzed for 48 hours at 4° C. using 10,000 MWCO.

### EXAMPLE 2

### Removal of LPS from the O-Ag Capsule

LPS was removed from the O-Ag capsule which had been purified as described above, using phase separation induced by 1% TRITON X-114 (Sigma-Aldrich) (Adam et al. (1995) Analylical Biochem. 225:321-327). The mixture was cooled overnight at 4° C. with stirring, or for ½ hour on ice at 4° C. with stirring, incubated for ½ at 37° C. and centrifuged at 25° C. for 1/2 hour at 1000 rpm and the upper phase removed and saved.

2% TRITON X-114 was then added to the lower phase, incubated at 37° C. for ½ hour on ice and the lower phases combined and incubated at 37° C. for ½ hour and centrifuged at 1000 rpm for ½ hour at 25° C. Any upper phase present was added to the previously saved sample. This process was repeated four times with the following centrifugation speeds as follows: 2nd purification step: 25° C., 1500 rpm, 1 hour (LPS: 3000 rpm, 1 hour); 3rd purification step: 25° C., 3000 rpm, 1.5 hours (LPS: 3000 rpm, 2 hours); 4th purification step: 25° C., 3000 rpm, 2 hours (LPS: 3000 rpm, 2-3 hours).

TRITON-X114 was then removed using a methanol-chloroform mixture and collecting the LPS-containing bottom phase (O-Ag capsule was in the top phase). The methanol-chloroform was then evaporated and the sample was dialyzed for 48 hours at 4° C. and lyophilized.

The amount of contaminating LPS was tested using the Limulus Amebocyte Lysate (LAL) Chromogenic Endotoxin Quantitation Kit (Pierce). The crude starting material registered 1.84×10⁸ endotoxin units (EU) per mg and the final LPS-purified material registered at 2.5×10³ EU/mg; one endotoxin unit is equivalent to 0.1 ng of Esherichia coli LPS per mL of solution.

Immune sera specific for the O-Ag capsule was generated from either Salmonella ser. Typhimurium or ser. Enteritidis by immunizing rabbits with three doses of LPS-purified capsular material; the first dose was 100 ug, and the last two doses were 50 ug.

### EXAMPLE 3

### Determining the Appropriate Dose of O-Ag Capsule

Four groups of 5 Balb/c mice (8 weeks old) are immunized intramuscularly (i.m.) with purified O-Ag capsule at Day 0 and Day 28. Each group receives a different dose: 1 μg, 5 μg, 25 μg or 40 μg. Blood samples are taken at Day 21 and 42 and ELISA is used to quantify the O-Ag capsule-specific antibody response in each group. If the response is poor, a third injection is administered. Adjuvants are evaluated for use with the vaccine.

### EXAMPLE 4

### Immunogenicity Study

Groups of 8 Balb/c mice (8 weeks old) receive two immunizations i.m. at Day 0 and Day 28 of the antigens listed in table 1 below. All mice are euthanized at Day 42.

TABLE 1

| Group | Antigen | Adjuvant |
|---|---|---|
| 1 | Purified O—Ag capsule | − |
| 2 | Purified O—Ag capsule | + |
| 3 | PBS (negative control) | − |
| 4 | Salmonella ser. Typhimurium SL7207 (aroA⁻) | − |
| | (positive control) | |

### Antibody Responses

Blood samples are taken at Day 0, 7, 14, 21, 28 and 35 and ELISA is used to quantify the O-Ag capsule-specific antibody responses. To characterize the nature of the immune response at Day 42, the isotype (IgG1 and IgG2a) of O-Ag-specific antibodies is determined. Secretory IgA (sIgA) is obtained on Day 42 by lavage of the small intestine and the levels of O-Ag-specific IgA are determined as above.

### Cytokine Profiles

To determine the capsule-specific cytokine responses at Day 42, spleens are collected from mice and ELISPOT assays are performed to detect the presence of cells producing IL-4, IFN-γ or IL-17. IFN-y production is indicative of a Th1-type immune response, whereas IL-4 is indicative of a Th2-type response. IL-17 production is indicative of a Th17 response.

### EXAMPLE 5

### Protection against Salmonella ser. Typhimurium Infection Using Purified O-Ag Capsule

To determine whether the O-Ag capsule is able to protect mice against Salmonella ser. Typhimurium infection, the following experiment is conducted. The following three groups of 10 Balb/c mice (8 weeks old) receive immunizations at Day 0 and Day 28:
Group 1—i.m. with vaccine antigen selected in Objective 2
Group 2—i.m. with PBS (negative control)
Group 3—intraperitoneal with 2×10⁵ CFU of S. Typhimurium SL7207 (positive control)

At Day 42, immunized mice are orally challenged with 1×10⁸ CFU S. Typhimurium 14028. If mice become infected, they are euthanized as soon as they show clinical signs of infection and/or >15% weight loss is recorded.

### Antibody Responses

To ensure that immunized mice have an O-Ag capsule-specific antibody response before challenge, blood samples are taken at Day 0 and Day 28 and ELISAs are performed as described previously. Samples taken at Day 50 are analyzed.

### Analyzing the Bacterial Load in Salmonella-Infected Mice

The spleen, liver, mesenteric lymph nodes and cecum are collected from all euthanized mice and homogenized in sterile PBS. Serial dilutions of homogenized tissue material are prepared in PBS and plated onto appropriate agar media. Bacterial load in the internal organs gives a measure of the severity of S. Typhimurium infection.

### EXAMPLE 6

### Protection Against ser. Enteritidis and Heidelberg using Salmonella ser. Typhimurium O-Ag Capsule

To determine if immunization with the O-Ag capsule is cross-protective, Example 5 is repeated with Salmonella ser. Enteritidis and ser. Heidelberg strains being used as the challenge strains on Day 42. At the end of each experiment, mice are euthanized and the number of Salmonella per organ (spleen, liver, mesenteric lymph nodes and cecum) is quantified.

In North America, the three most common S. enterica subsp. enterica serovars that cause human infections are Typhimurium, Enteritidis, and Heidelberg. Cross-protection between S. enterica serovars provides a highly desirable traveler's vaccine.

### EXAMPLE 7

### Influence of YihVW on Gene Expression of the vihU-yshA Operon

To determine the role of yihV and yihW as transcriptional modifiers, the following experiment was conducted. To confirm the function of YihV and YihW on gene expression of the O-Ag capsule operon (yihU-yishA), a ΔyihVW strain was constructed using the λ-red recombination system (Datsenko, et al. (2000) Proc. Natl. Acad. Sci 97:6640-6645). Using the methods detailed above, a chloramphenicol marker was inserted into the region in the chromosome where yihVW would normally occur. A pBR322 plasmid construct was also generated that contained yihVW, where yihVW expression would occur from the native promoter. When this plasmid was transformed into the various S. Typhimurium strains and the resulting transformants were grown in culture, the YihV and YihW genes were overexpressed, indicating that these genes act as transcriptional modifiers and deletion of one or both of these genes or portions thereof, can serve to boost expression of the O-Ag capsule.

The sequence of the yihVW region is shown in FIGS. 4A-4B. The portion bolded in the figure represents the deletion present in the yihVW mutant produced herein (corresponding to positions 96-1822).

### EXAMPLE 8

### Gene Expression Studies

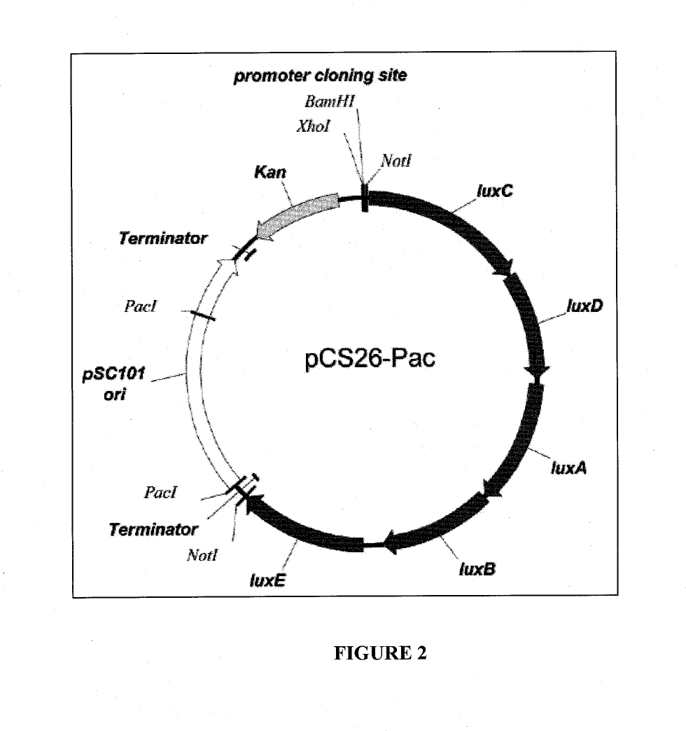

It has been shown that the yihU promoter is activated during S. Typhimurium infection of mice (White et al. (2008) Infect Immun. 76:1048-1058). This indicates the O-Ag capsule is expressed during infection. To study O-Ag capsule production at the cellular level, expression of the yihU and yihV promoters were monitored under different conditions using a luciferase assay. For this, a pCS26-Pac plasmid (Bjarnason et al. (2003) J. Bacteriol. 185(16): 4973-4982, FIG. 2) was used. Plasmid pCS26-Pac is a low copy number plasmid containing luxCDABE of Photorhabdus luminescens. The resulting plasmid contained PyihU or PyihV cloned in front of luxCDABE. Activation of transcription from either promoter would lead to the expression of luxCDABE and production of light. Thus the intensity of luciferase produced is a clear indication of the promoter activity.

For luciferase assays, the strains shown in Table 2 were constructed, so that each strain had a pCS26-Pac plasmid containing PyihU or PyihV. Luciferase was measured using a Wallac-Victor X³ multi-label plate reader (Perkin-Elmer). The various reporter strains were inoculated into wells of a 96-well plate, and light production was assessed during growth for 48 h at 30° C. in three different growth media: EPS media, EPS media with 2,2′-dipyridyl (an iron chelater), and 1% Tryptone.

TABLE 2
S. Typhimurium strains used in the luciferase assays.

| Strain | Media | | |
|---|---|---|---|
| S. Typhimurium ΔbcsA + PyihU::lux | EPS | EPS media + 2,2′- | 1% Tryptone |
| | media | dipyridyl | |
| S. Typhimurium ΔbcsA + PyihV::lux | EPS | EPS media + 2,2′- | l% Tryptone |
| | media | dipyridyl | |
| S. Typhimurium ΔbcsA pBR322-yihVW + PyihU::lux | EPS | EPS media + 2,2′- | 1% Tryptone |
| | media | dipyridyl | |
| S. Typhimurium ΔbcsA pBR322-yihVW + PyihV::lux | EPS | EPS media + 2,2′- | 1% Tryptone |
| | media | dipyridyl | |
| S. Typhimurium ΔbcsA ΔyihVW + PyihU::lux | EPS | EPS media + 2,2′- | 1% Tryptone |
| | media | dipyridyl | |
| S. Typhimurium ΔbcsA ΔyihVW + PyihV::lux | EPS | EPS media + 2,2′- | 1% Tryptone |
| | media | dipyridyl | |
| S. Typhimurium ΔbcsA ΔyihVW pBR322-yihVW + PyihU::lux | EPS | EPS media + 2,2′- | 1% Tryptone |
| | media | dipyridyl | |
| S. TyphimuriumΔbcsAΔyihVWpBR322-yihVW + PyihV::lux | EPS | EPS media + 2,2′- | 1% Tryptone |
| | media | dipyridyl | |

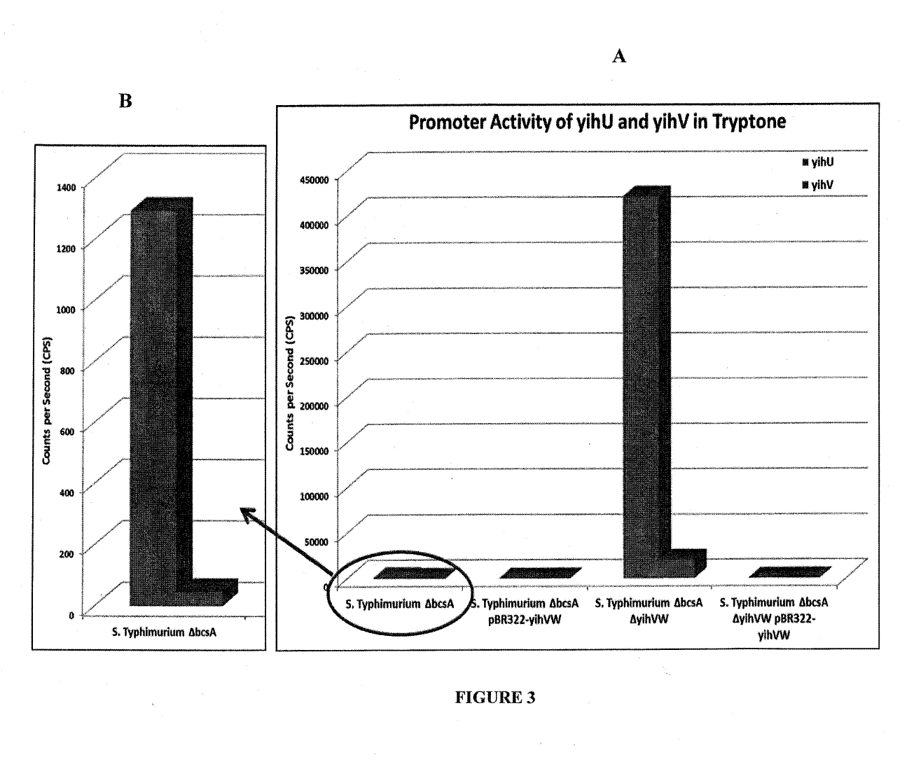

In all four strains: S. Typhimurium ΔbcsA, S. Typhimurium ΔbcsA pBR322-yihVW, S. Typhimurium ΔbcsA ΔyihVW, and S. Typhimurium ΔbcsA ΔyihVWpBR322-yihVW promoter activity of yihU was higher than that of yihV. The two strains with the plasmid (pBR322-yihVW) had very low levels of light production (FIGS. 3A and 3B). Both S. Typhimurium ΔbcsA and S. Typhimurium ΔbcsA ΔyihVW strains had the highest activity of PyihU and PyihV in 1% Tryptone, while the lowest activity was in EPS media containing 2,2′-dipyridyl. In 1% Tryptone, S. Typhimurium ΔbcsA ΔyihVW PyihU had 1000 times more activity (i.e. light production) than S. Typhimurium ΔbcsA PyihU. Out of all eight strains used in this assay, PyihU in S. Typhimurium ΔbcsA ΔyihVW had the highest activity and was best expressed in 1% Tryptone.

The fact that the ΔyihVW strain had the highest activity indicates that in the absence of YihVW, PyihU has higher expression. This evidences that one or both of YihVW are repressors and deletion of one or both of these two repressors increases PyihU activity. FIG. 3A shows the PyihU activity of all four strains grown on 1% Tryptone. As noted above, the S. Typhimirum ΔbcsA ΔyihVW strain had the highest PyihU activity, while the two strains with the plasmid (pBR322-yihVW) had the lowest PyihU activity. Over-expression of yihVW in plasmids decreases the promoter activity of yihU. This further supports the observation that YihVW are repressors of the yihU-yihO operon. Thus, based on gene expression levels observed in this assay, the use of knockout mutants of S. Typhimurium, such as ΔbcsA ΔyihVW, greatly enhances O-Ag capsule production and allows purification of more O-Ag capsule.

In order to determine whether the gene expression observed in liquid culture was also seen on agar, the following experiment was conducted. For this, streak plates of S. Typhimurium ΔbcsA, S. Typhimurium ΔbcsA pBR322 -yihVW, S. Typhimurium ΔbcsA ΔyihVW, and S. Typhimurium ΔbcsA ΔyihVW pBR322-yihVW were made on LB agar. Light production by individual colonies was observed with an IVIS Lumina II Bioluminescent Imager (Perkin-Elmer). Again, higher PyihU expression was observed in the S. Typhimurium ΔbcsA ΔyihVW strain, as compared to S. Typhimurium ΔbcsA. The two strains with the plasmid (pBR322-yihVW) had no detectable level of light production.

Thus, immunogenic compositions and methods of making and using the same for treating and preventing Salmonella infection using O-Ag capsules from S. Enteritidis NTS serovars are described. Although preferred embodiments of the subject invention have been described in some detail, it is understood that obvious variations can be made without departing from the spirit and the scope of the invention as defined by the appended claims.

1. A method of preparing an NTS O-Ag capsule preparation comprising purifying the O-Ag capsule from an S. enterica NTS serovar, wherein the O-Ag capsule is substantially free of co-expressed cellulose and LPS.

2. The method of claim 1, wherein the method comprises:
(a) providing a cellulose-deficient S. enterica NTS serovar mutant;
(b) isolating cell surface components from the S. enterica NTS serovar, wherein the cell surface components comprise the O-Ag capsule;
(c) applying the cell surface components to an anion exchange chromatography column under conditions whereby fractions comprising the O-Ag capsule are eluted;
(d) applying O-Ag capsule-containing fractions to a size-exclusion chromatography column under conditions whereby fractions comprising the O-Ag capsule are eluted;
(e) collecting fractions that include the O-Ag capsule;
(f) performing phase separation on the O-Ag capsule-containing fractions under conditions that separate LPS from the O-Ag capsule;
to provide O-Ag capsule substantially free of co-expressed cellulose and LPS.

3. The method of claim 2, wherein step (f) is performed using a polyethylene glycol detergent.

4. The method of claim 1, wherein the amount of LPS remaining in the final product is under 2×10⁵ EU/mg.

5. The method of claim 1, wherein the S. enterica NTS serovar is selected from serovar Enteritidis (S. Enteritidis), serovar Typhimurium (S. Typhimurium) or serovar Heidelberg (S. Heidelberg).

6. The method of claim 5, wherein the S. enterica NTS serovar is S. Typhimurium.

7. A composition comprising a pharmaceutically acceptable vehicle and (a) an immunogenic S. enterica NTS O-Ag capsule, wherein the O-Ag capsule is substantially free of co-expressed cellulose and LPS; (b) an immunogenic fragment of (a), or (c) antibodies reactive with the O-Ag capsule.

8. A composition comprising an immunogenic S. enterica NTS O-Ag capsule, wherein the S. enterica NTS O-Ag capsule is prepared by the method claim 1.

9. The composition of claim 7, wherein the S. enterica NTS serovar is selected from serovar Enteritidis (S. Enteritidis), serovar Typhimurium (S. Typhimurium) or serovar Heidelberg (S. Heidelberg).

10. The composition of claim 9, wherein the S. enterica NTS serovar is S. Typhimurium.

11. Original) A method of producing an immunogenic composition comprising (a) providing a purified, immunogenic S. enterica NTS O-Ag capsule; and (b) combining said purified O-Ag capsule with a pharmaceutically acceptable vehicle.

12. The method of claim 11, wherein the S. enterica NTS 0-Ag capsule is prepared by purifying the O-Ag capsule from an S. enterica NTS serovar, wherein the O-Ag capsule is substantially free of co-expressed cellulose and LPS.

13. A method of treating or preventing an S. enterica NTS infection in a vertebrate subject comprising administering to the subject a therapeutically effective amount of a composition according to claim 7.

14. A method of reducing the amount of S. enterica NTS in the intestinal tract of a vertebrate subject comprising administering to the subject a therapeutically effective amount of a composition according to claim 7.

15. A method of detecting S. enterica NTS antibodies in a biological sample, comprising:
(a) reacting the biological sample with an immunogenic S. enterica NTS O-Ag capsule, under conditions which allow NTS antibodies, when present in the biological sample, to bind to the capsule to form an antibody/antigen complex; and
(b) detecting the presence or absence of the complex, and thereby detecting the presence or absence of S. enterica NTS antibodies in the sample.

16. An immunodiagnostic test kit for detecting S. enterica NTS infection, the test kit comprising an immunogenic S. enterica NTS O-Ag capsule and instructions for conducting the immunodiagnostic test.

17. A polynucleotide encoding an S. enterica NTS serovar mutant, wherein said mutant comprises a deletion of all or a portion of the yihV and/or yihW genes of the 0-Ag capsule operon, wherein when the polynucleotide is expressed, O-Ag capsule production is enhanced as compared to O-Ag capsule production when yihVW remains intact.

18. The polynucleotide of claim 17, wherein said mutant comprises a deletion of the nucleotide sequence encoding the DNA-binding region of YihW.

19. The polynucleotide of claim 18, further comprising a deletion of the gene coding for cellulose synthase.

20. The polynucleotide of any one of claims 17 19 claim 17, wherein the S. enterica NTS serovar is selected from serovar Enteritidis (S. Enteritidis), serovar Typhimurium (S. Typhimurium) or serovar Heidelberg (S. Heidelberg).

21. The polynucleotide of claim 20, wherein the S. enterica NTS serovar is S. Typhimurium.

22. A recombinant construct comprising;
(a) the polynucleotide of claim 17; and
(b) control elements that are operably linked to said polynucleotide whereby coding sequences in said polynucleotide can be transcribed and translated in a host cell.

23. A host cell transformed with the recombinant construct of claim 22.

24. A method of producing an O-Ag capsule comprising:
(a) providing a population of host cells according to claim 23; and
(b) culturing said population of cells under conditions whereby the O-Ag capsule is produced.

25. The method of claim 24, further comprising purifying the produced O-Ag capsule to provide an O-Ag capsule preparation, wherein O-Ag capsule is purified under conditions wherein the O-Ag capsule preparation is substantially free of co-expressed cellulose and LPS.

26. A conjugate comprising an S. enterica NTS O-Ag capsule, wherein the O-Ag capsule is substantially free of co-expressed cellulose and LPS conjugated to a carrier molecule.

27. A composition comprising the conjugate of claim 26, and a pharmaceutically acceptable vehicle.


SEQUENCE LISTING (7 sequences)

SEQ ID NO: 1 - DNA, 70 bp, Artificial Sequence
  Synthetic primer
ttcgtgaaattaaaatgagcacatcgaaaatgcttgaggaatgaccatgggtgtaggctggagctgcttc

SEQ ID NO: 2 - DNA, 70 bp, Artificial Sequence
  Synthetic primer
ttggccggataaagcgctgacgcgaccctccggcgcaagggcgcttgtcacctccttagttcctattccg

SEQ ID NO: 3 - DNA, 22 bp, Artificial Sequence
  Synthetic primer
gcacatcgaaaatgcttgagga

SEQ ID NO: 4 - DNA, 20 bp, Artificial Sequence
  Synthetic primer
atatcgcctgcatcacagcg

SEQ ID NO: 5 - DNA, 1873 bp, S. Typhimurium str. LT2
  misc_feature: S. enterica NTS serovar (S. Typhimurium str. LT2) yihVW region
tgtaatttatagtcaaaaaattgacagccgtcactttttaaacatttcgtgaaattaaaatgagcacatcgaaaatgcttgaggaatgaccatggttcgtattgcttgtgtaggtattaccgtgatggatcgcatctattacgtggaaggattaccgacagaagggggtaaatatgtggcgaagcgctatacggaagtgggcggcgggcctgcggcgaccgctgccgtggctgcggcgaaattgggcgcacaggtggactttattggccgtgtgggcgatgacgacaccggaaacagcctgctggcggaactggaatccctgggggtaaatacccgctatacccgccgctacacgcaggccatgtcgtcgcagtcagcgattatggtggatgccaaaggagagcggattatcgttaattatcccagcccggatttattacctgacgccgactggcttaacgacatcgatttttcgcagtgggatgtggtgttggcggatgtgcgctggcatgacggcgcaaaacaggcgtttacgctggcgcgtcaggctggcgtcatgactgttcttgatggcgatattacgccgcaggatattagcgagctggtggcgttaagcgaccacgccgccttctccgagccagggctggcacgcctgacggggatgagcgaagccattgatgcgctaaaaaaagcacaaatgctcacaaatggacatgtctatgtcacgcgaggcagtgaaggctgcaactggttggaaaaagcggcggtacgccaccaaccgggttttaccgtggaggtggtggatactactggcgcgggcgatgtttttcatggcgcactggcatttggcctggcaagcggatacgccatcgaagaggctgtcagattcgccagcggcgttgccgcgctgaagtgtacgcgcccgggtggccgggcgggtatccctgattgtgagcaaacccgatctttcttgtcactttttgtataatatgagcagtaggaagcttttagaggaatgctcatgagtcttaccgaactgaccggtaatccgcgacacgatcggctgttaatgctaattgatgagcgcggctatatgaatatcgatgagttagccagtttgttggaggtctctacccagacggttcgccgcgatattcgtaaattaagcgagcaggggctgattacgcgtcatcacggcggcgctggcagagcgtccagtgtggtcaataccgcctttgagcagcgagaggtctcctggacgcaagagaaaaaagccattgcggaagccgtggctgactatattccggatggctccacgatatttatcacgattggaacgacggttgagcaggtggcgcgcgcgctgctcaaccataatcatctgcgcattatcaccaatagcctgcgcgtcgcgcacattctttataataaccctcgttttgaagtgatggtgcctggcggcacgctgcgcccgcataatagcggcattatcgggccatccgcggcggcgtttgtggcggggttccgggctgactatctcgtcaccagcgtaggcgcgatagagagcgatggcgcgttactggagtttgatgtcaacgaagccagcgtagtgaaaaccatgatggcccactcgcgacatattttgctggccgccgatcatacgaaataccatgcttctgcggcggttgagatcggcaatgtgtcacagattaccgctctgtttacggacgagtatcccggtccggcattacagaatttacttcaatcccagcaaatcgaagtggttcaggtgagtccttcgctggacgacgccgtttcagcgtgacaagcgcccttgcgccggagggtcgcgtcagcgctttatccggccaat

SEQ ID NO: 6 - protein, 298 aa, S. Typhimurium str. LT2
  misc_feature: amino acid sequence of YihV encoded by the nucleotide sequence shown in SEQ ID NO 5
MVRIACVGITVMDRIYYVEGLPTEGGKYVAKRYTEVGGGPAATAAVAAAKLGAQVDFIGRVGDDDTGNSLLAELESLGVNTRYTRRYTQAMSSQSAIMVDAKGERIIVNYPSPDLLPDADWLNDIDFSQWDVVLADVRWHDGAKQAFTLARQAGVMTVLDGDITPQDISELVALSDHAAFSEPGLARLTGMSEAIDALKKAQMLTNGHVYVTRGSEGCNWLEKAAVRHQPGFTVEVVDTTGAGDVFHGALAFGLASGYAIEEAVRFASGVAALKCTRPGGRAGIPDCEQTRSFLSLFV

SEQ ID NO: 7 - protein, 267 aa, S. Typhimurium str. LT2
  misc_feature: amino acid sequence of YihW encoded by the nucleotide sequence shown in SEQ ID NO 5
MSLTELTGNPRHDRLLMLIDERGYMNIDELASLLEVSTQTVRRDIRKLSEQGLITRHHGGAGRASSVVNTAFEQREVSWTQEKKAIAEAVADYIPDGSTIFITIGTTVEQVARALLNHNHLRIITNSLRVAHILYNNPRFEVMVPGGTLRPHNSGIIGPSAAAFVAGFRADYLVTSVGAIESDGALLEFDVNEASVVKTMMAHSRHILLAADHTKYHASAAVEIGNVSQITALFTDEYPGPALQNLLQSQQIEVVQVSPSLDDAVSA